(19) United States
(12) Patent Application Publication
(10) Pub. No.: US 2008/0199885 A1
(43) Pub. Date: Aug. 21, 2008

(54) Overexpression of mammaglobin B in ovarian and endometrial tumors - a new diagnostic and therapeutic marker

(75) Inventor: Alessandro D. Santin, Little Rock, AR (US)

Correspondence Address: HUGH MCTAVISH;MCTAVISH PATENT FIRM, 429 BIRCHWOOD COURTS, BIRCHWOOD, MN, 55110, US

(73) Assignee: The Board of Trustees of the University of Arkansas, Little Rock, AR (US)

(21) Appl. No.: 11/709,014

(22) Filed: Feb. 21, 2007

(51) Int. Cl. A61K 38/02; A61P 35/00; G01N 33/53

(52) U.S. Cl. 435/7.8; 514/12

(57) ABSTRACT

The invention involves the discovery that the mammaglobin B gene is the single most overexpressed gene in primary ovarian serous papillary cancer over normal ovarian epithelium among over 14,000 genes tested. It is expressed over 800-fold higher in primary ovarian tumors than normal ovarian epithelium. Mammaglobin B gene expression was detected in endometrioid, mucinous, undifferentiated, serous papillary, clear cell, and mixed histology ovarian tumors. The protein can be found in blood and ascites fluid, and a simple blood test for the presence of mammaglobin B protein can provide early detection of ovarian and other cancers. The invention provides a method of screening for cancer involving detecting mammaglobin B in a fluid sample.

### BACKGROUND

Ovarian cancer remains the most lethal gynecologic tumor in the United States and in the Western world, with 22,220 new cases expected in the USA in 2005 and 16,210 deaths [1]. Serous papillary adenocarcinoma (OSPC) is the most common histologic subtype of ovarian cancer, accounting for nearly 50% of all ovarian epithelial tumors [2]. Although aggressive cytoreductive surgery followed by adjuvant platinum-based chemotherapy is able to induce clinical responses in the majority of ovarian cancer patients, most of these women eventually relapse and die from the development of chemotherapy-resistant disease [2]. The identification of novel molecular markers useful for the early diagnosis and therapy of ovarian cancer patients remains a high priority.

A better understanding of the molecular circuitry in normal ovarian tissues and in ovarian cancer will likely provide promising targets for novel therapeutic strategies. Consistent with this view, with the goal of identifying genes with a differential pattern of expression between ovarian tumors and normal ovarian cells and to use this knowledge for the development of novel diagnostic and therapeutic markers against this disease, several groups including our own have recently used high throughput gene array technologies to compare the expression profiles of ovarian cancer to those of normal ovaries [3-6, 13, 14]. Using different sets of probes, ovarian cancer genetic fingerprints have been studied in flash-frozen tumor biopsies as well as primary and/or established ovarian cancer cell lines [3-6, 13, 14]. Similarly, a variety of sources of normal cells for comparison with tumors, including whole ovary samples, ovarian surface epithelium exposed to short-term culture or immortalized normal ovarian cell lines, have been used as controls [3-6, 13, 14, 21]. All these approaches, however, have potential drawbacks including (i) the presence of different percentages of contaminant RNA derived from the stromal and inflammatory components present in flash-frozen biopsies, (ii) changes in gene expression related to the manipulation and removal of epithelial ovarian cells from their microenvironment as well as the potential growth of only a subset of epithelia cells during short-term in vitro culture and (iii) acquirement of new genetic mutations during long-term in vitro growth.

Mammaglobin B is a uteroglobin gene family member isolated in the rat uterus [28] and human endometrium [29] that encodes a 95-amino acid secreted protein of 10 kDa sharing very high homology with mammaglobin A, a promising diagnostic marker for breast cancer. Mammaglobin B has been previously reported to be overexpressed in primary breast cancer tissues and in occult breast metastases [68, 69], and its mRNA has been detected by RT-PCR to detect breast cancer micrometastases in lymph nodes, bone marrow, and circulation [52, 53, 70]. Mammaglobin B is also known as mammaglobin 2 or MGB-2.

New markers and methods to provide early detection of ovarian and other cancers are needed. New tools for therapeutic treatment of ovarian and other cancers are needed.

### SUMMARY

The invention involves the discovery that the mammaglobin B gene is the single most overexpressed gene in primary ovarian serous papillary cancer over normal ovarian epithelium among over 14,000 genes tested. It is expressed over 800-fold higher in primary ovarian tumors than normal ovarian epithelium. This is the first demonstration of overexpression of mammaglobin B in early stage ovarian tumors. Mammaglobin B transcript was detected at above the 95th percentile of its normal ovarian tissue expression level in 100% of ovarian cancer specimens tested. Mammaglobin B gene expression was detected in endometrioid, mucinous, undifferentiated, serous papillary, clear cell, and mixed histology ovarian tumors. By histological staining, mammaglobin B protein was detected in a large fraction of ovarian tumors as well. So it is a suitable early stage marker for all types of ovarian malignancies.

Mammaglobin B is a secreted protein, and overexpression of its gene transcript appeared more extreme in ovarian tumors than overabundance of the protein. Thus, the protein can be found in blood and ascites fluid, and a simple blood test for the presence of mammaglobin B protein can provide early detection of ovarian and other cancers.

The extreme overexpression of mammaglobin B in ovarian tumors and its lack of significant expression in normal ovarian tissue and low level of expression in other tissues makes it a good target for targeted cancer therapy with antibodies and other agents that bind specifically to mammaglobin B.

Accordingly, the invention provides a method of screening for cancer involving (a) obtaining a fluid sample (typically blood or ascites fluid) from a mammal; (b) contacting the fluid sample with a mammaglobin B binding protein to bind mammaglobin B in the sample; and (c) quantifying binding of the binding protein to mammaglobin B to detect the presence of mammaglobin B above a threshold level, wherein the presence of mammaglobin B above the threshold level indicates presence of cancer in the mammal. The method is particularly suited for screening for ovarian cancer.

Another embodiment of the invention provides a kit to detect mammaglobin B in a biological fluid sample containing: a mammaglobin B binding protein (e.g., an anti-mammaglobin B antibody); and a means for detecting binding of the mammaglobin B binding protein to mammaglobin B in a biological fluid sample.

Another embodiment of the invention provides a method of treating cancer involving: inoculating an individual (a mammal) suffering from or at risk of cancer with a mammaglobin B peptide, wherein the inoculation elicits an immune response in the individual against cells expressing mammaglobin B.

Another embodiment of the invention provides an immunogenic composition comprising a mammaglobin B peptide and an adjuvant.

Another embodiment of the invention provides an immunogenic composition comprising dendritic cells loaded with a mammaglobin B peptide.

Another embodiment of the invention provides a humanized anti-mammaglobin B antibody.

Another embodiment of the invention provides an anti-cancer agent comprising a mammaglobin B binding protein coupled to a cytotoxic agent.

Another embodiment of the invention provides a method of treating cancer comprising administering to a person suffering from cancer an anti-cancer agent containing a mammaglobin B binding protein coupled to a cytotoxic agent.

Another embodiment of the invention provides a method of treating cancer comprising administering to a person suffering from cancer an anti-mammaglobin B antibody.

### BRIEF DESCRIPTION OF THE DRAWINGS

FIG. 1 is a drawing of a lateral flow immunoassay device for determining the presence or absence of mammaglobin B in a sample.

FIG. 2. Dendrogram resulting from unsupervised cluster analysis readily distinguishes between gene expression profiles of OSPC and HOSE.

FIG. 3. Quantitative RT-PCR and microarray expression analysis of CLDN7, CXCR-4, KLK6, MUC1, MUC16/CA125 and SCGB2A1 genes differentially expressed between OSPC and HOSE. q-RT-PCR data were highly correlated to the microarray data (P<0.001).

FIG. 4. Representative immunohistochemical staining for mammaglobin 2 (upper panels), MUC1 (middle panels) and CXCR4 (lower panels) antigens. Left panels: normal controls showing no staining for mammaglobin 2, MUC1 and CXCR4. Right panels: OSPC displays a strong membranous and/or cytoplasmic staining for mammaglobin 2, MUC1 and CXCR4. Original magnification 400×.

FIG. 5. This figure shows the boxplots of the RT-PCR values in ln scale. Boxplots depict the mean and the median, the first and the third quartile (the box).

FIG. 6. Representative patterns of mammaglobin B immunoreactivity in primary EOCs, omental metastases, benign cystadenoma and normal ovarian tissues. Mammaglobin B was undetectable in 7/7 normal ovaries (A), in 23/26 benign cystadenomas (B), 7/8 borderline tumors (C) and 24/28 serous-papillary omental metastases (F). Strong to moderate mammaglobin B cytoplasmic staining was detectable in 70% of endometrioid (E), 50% of mixed (G), 30% of serous-papillary (H) and 50% of clear-cell (D) primary EOCs.

### DETAILED DESCRIPTION

### Definitions

The term “peptide” as used herein includes full-length proteins and a segment of the full-length protein.

The term “peptide-loaded dendritic cell” as used herein refers to a dendritic cell presenting a peptide on its surface in a manner effective to amplify T cells that specifically recognize the peptide. The dendritic cell may become loaded with the peptide by directly binding the peptide from the medium on its surface, or by processing the peptide intracytoplasmically before presenting the peptide. Processing the peptide may include proteolytically generating the presented peptide from a longer peptide. T cells that specifically recognize the peptide are T cells that kill autologous cells pulsed with the peptide or proliferate in response to contacting autologous cells pulsed with the peptide.

The term “humanized antibody” refers to an antibody from a non-human source, typically, a mouse monoclonal antibody, in which some of the protein sequence of the non-human antibody is replaced by human antibody protein sequence in order to reduce the immune response in a human to the antibody.

The term “mammaglobin B binding protein” refers to a protein that binds specifically to mammaglobin B.

### Description:

The invention involves the discovery that the mammaglobin B gene is the single most overexpressed gene in epithelial ovarian tumors among over 14,000 genes tested, with an expression level of over 800-fold higher than the baseline expression in normal ovarian tissue. The mammaglobin B protein is also detected in ovarian cancer tumors by antibody staining. And mammaglobin B is a secreted protein, so it is found in blood and ascites fluid when it is expressed in ovarian cancer. It is shown here for the first time that mammaglobin B is expressed at these very elevated levels even in primary ovarian tumors, not just in metastasized cancers. This makes it suitable as an early sign of cancer.

Thus, one embodiment of the invention provides a method of screening for cancer involving (a) obtaining a fluid sample (typically blood or ascites fluid) from a mammal; (b) contacting the fluid sample with a mammaglobin B binding protein to bind mammaglobin B in the sample; and (c) quantifying binding of the binding protein to mammaglobin B to detect the presence of mammaglobin B above a threshold level, wherein the presence of mammaglobin B above the threshold level indicates the presence of cancer. The method is particularly suited for screening for ovarian cancer. Preferably the method is used to screen for ovarian cancer, but evidence suggests that mammaglobin B is expressed in other types of cancer, and so the method can be used to detect any type of cancer whose cells secrete mammaglobin B.

The mammal in a preferred embodiment is a human. In one embodiment the mammal is a female.

The fluid sample is typically blood or ascites fluid. The term “blood” in this context includes processed or fractionated blood, including plasma and serum.

In particular embodiments, the cancer is a gynecological malignancy, e.g., uterine or ovarian cancer.

In particular embodiments, the cancer is ovarian cancer.

In other embodiment, the cancer is uterine cancer. The data below in Example 2 shows that the mammaglobin B gene is greatly overexpressed in ovarian cancer endometrioid type, which is identical to endometrial cancer arising in the uterus.

In particular embodiments, the cancer is breast cancer. In other embodiments the cancer is not breast cancer.

In a preferred embodiment, the cancer detected has not metastasized.

The step in the method of quantifying binding of the binding protein to mammaglobin B to detect the presence of mammaglobin B above a threshold level may involve giving a numerical result for the concentration of mammaglobin B in the fluid sample, or it may give a qualitative result that mammaglobin B is or is not present above the threshold level.

The mammaglobin B binding protein is typically an antibody that specifically binds mammaglobin B. But it could be a protein that is not an antibody—i.e. is not produced originally by a vertebrate as an immune response to a substance—that specifically binds mammaglobin B. For instance, it could be a peptide or protein selected from a phage display library for the property of binding to a mammaglobin B peptide.

A preferred detection method is an ELISA assay. In one type of ELISA, a mammaglobin B binding protein is immobilized on the surface of a solid substrate, such as a multi-well plate. Serum or other fluid from a mammalian subject being screened for cancer is incubated in the well to contact the immobilized binding protein. Any mammaglobin B present in the fluid binds to the binding protein. The fluid sample is then removed and the surface is washed. Then the surface is contacted with free mammaglobin B binding protein coupled to an enzyme that acts on a chromogenic substrate, such as peroxidase. The enzyme-linked mammaglobin B binding protein binds to any mammaglobin B held on the substrate by the immobilized mammaglobin B binding protein to form an immobilized antibody-antigen-antibody-enzyme sandwich. The substrate is then washed to remove unbound mammaglobin B binding protein-enzyme complex. The well of the multiwell plate is then incubated with a substrate that changes color when acted upon by the enzyme. If the coupled enzyme is peroxidase, it may for instance be incubated with hydrogen peroxide and tetramethylbenzidene as a chromogen.

Alternatively, the free mammaglobin B binding protein could be coupled to, e.g., biotin, and the substrate with the bound biotin-coupled binding protein can be incubated with streptavidin-enzyme complex to immobilize the enzyme.

Other means of detecting bound protein are known in the art, including coupling the secondary antibody to a fluorescent moiety or to an enzyme that activates a chemiluminescent substrate (e.g., luciferase, which acts on luciferin in the presence of ATP).

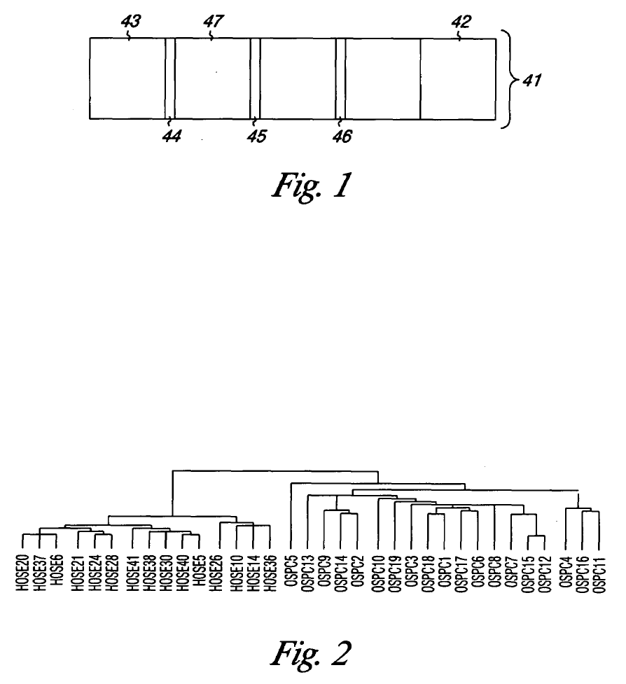

A lateral flow immunoassay is another method of carrying out the method. Lateral flow immunoassays are perhaps most commonly used to test for pregnancy by detecting pregnancy hormones with antibodies. One embodiment is shown in FIG. 1. Lateral flow immunoassay device 41 is shown. To detect mammaglobin B in a fluid sample, a dye-conjugated antibody against mammaglobin B is mixed with the sample. The dye-conjugated antibody may be immobilized on gold or latex particles. This amplifies the signal since multiple antibody and dye molecules are on the same particle. In the embodiment of FIG. 1, the device 41 of a kit of the invention includes a substrate 47 that can be, for instance, nitrocellulose. A dye-conjugated anti-mammaglobin B antibody is impregnated at area 44 of a substrate 47. A fluid sample is placed at area 43 of the substrate 47. The sample can be placed directly in contact with the dye-conjugated antibody or may migrate to contact the antibody. The sample contacts the dye-conjugated antibody in area 44, and then by lateral fluid flow, the sample and the dye-conjugated antibody migrate toward an absorbent pad 42 at the other end of the device 41. Two strips of immobilized antibody are located at positions 45 and 46. The first strip 45 contains immobilized antibody that recognizes the same antigen as the dye-conjugated antibody. Thus, if the dye-conjugated antibody has bound mammaglobin B antigen, this immobilized antibody also binds to the bound mammaglobin B, and the dye-conjugated antibody is bound to the first strip 45 as a dye-conjugated antibody-antigen-immobilized antibody sandwich. A second strip 46 contains immobilized anti-Fc-antibody that recognizes the Fc portion of the dye-conjugated antibody. If the sample contains no antigen recognized by the dye-conjugated antibody, the dye-conjugated antibody has no bound antigen and thus does not bind as a sandwich to the immobilized antibody of the first strip 45 and it passes by the first strip 45. It then reaches the second strip 46 where it is bound by the anti-Fc antibody. Thus, when the sample contains antigen recognized by the dye-conjugated antibody, in this case mammaglobin B, the dye-conjugated antibody accumulates on the first strip 45. If the sample does not contain the antigen, the dye-conjugated antibody accumulates on the second strip 46. The dye can be detected visually as a colored band. Or if the dye is fluorescent or chemiluminescent, it can be detected by light emission.

Bioassayworks, LLC, (Ijamsville, Md.; www.bioassayworks.com) prepares gold-antibody conjugates and lateral flow immunoassay kits.

Lateral flow immunoassays are best suited to give a simple positive or negative result for the presence of an antigen, rather than a numerical value for the quantity of an antigen. However, since the difference in expression of mammaglobin B between normal and malignant ovarian tissue is so large, this type of test kit may be suitable for distinguishing between abnormal and normal quantities of mammaglobin B.

Another embodiment of the invention provides a method of treating cancer involving inoculating an individual suffering from or at risk of cancer with a mammaglobin B peptide, wherein the inoculation elicits an immune response in the individual against mammaglobin B.

The most powerful way to do this is to use peptide-loaded dendritic cells. Thus, in one embodiment the peptide is on dendritic cells. Dendritic cells are effective antigen-presenting cells. They are particularly adept at stimulating naive T cells. Dendritic cell function is reviewed in references [71] and [72]. Peptide-loaded dendritic cells are created by contacting dendritic cells with a purified peptide. This may be done by any suitable method, including mixing the dendritic cells with the purified peptide directly, mixing dendritic cells with purified peptide in liposomes, and expressing the purified peptide from a recombinant nucleic acid in the dendritic cells. The purified peptide could be where mammaglobin B or a peptide segment of mammaglobin B or a mixture of mammaglobin B peptide segments.

For example, to isolate dendritic cells from a cancer patient, the patient can undergo leukopheresis using a COBE separator. Peripheral blood leukocytes (PBL) from the patients are used for generation of dendritic cells (DC). Monocyte-derived DC are cultured in AIM-V (Gibco-BRL) supplemented with GM-CSF and IL-4 [63]. After 5 days' culture, DC maturation is induced by addition of TNFα, IL-1β, and GPE₂ [63]. Mature DC are incubated for 1-2 hours at 37° C. with 50 μg/ml of a mammaglobin B peptide. The culture medium is AIM-V plus 5% human AB serum (Gemini Bioproducts). No IL-2 is added. The DC are then washed twice to remove unbound peptides. The DC are then suspended in PBS supplemented with 10% autologous serum, and infused intravenously into the patient over a period of one hour.

In specific embodiments of the method of treating cancer involving inoculating an individual with a mammaglobin B peptide, the peptide is associated with an adjuvant.

The individual inoculated is a mammal. The individual is generally a human, but the method can be used on pets or tested in non-human mammals.

Preferably the inoculating slows or reverses the growth of the cancer.

In some embodiments, the individual is in remission from cancer and the inoculating decreases the risk of recurrence of the cancer.

In specific embodiments, the cancer is a gynecological malignancy, preferably ovarian cancer. In specific embodiments, the gynecological malignancy is uterine cancer.

In specific embodiments, the cancer is breast cancer. In other embodiments it is not breast cancer.

The invention also involves a method of treating cancer involving administering to a mammal suffering from cancer an anti-mammaglobin B antibody.

The antibodies used in the present invention can be prepared by a variety of methods [See 66, Chapter 2]. For example, cells expressing mammaglobin B can be administered to an animal to induce the production of sera containing polyclonal antibodies. In a preferred method, mammaglobin B is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. [73-76.] In general, such procedures involve immunizing an animal (preferably a mouse) with polypeptide or, more preferably, with a secreted polypeptide-expressing cell. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention. After fusion, the resulting hybridoma cells are selectively maintained in HAT medium, and then cloned by limiting dilution as described in [67]. The hybridoma cells obtained through such a selection are then assayed to identify clones which secrete antibodies capable of binding the polypeptide.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See for review [77-85].) It will be appreciated that Fab and F(ab′)2 and other fragments of the antibodies of the present invention may be used according to the methods disclosed herein, particularly when coupled to cytotoxic agents. Such fragments are typically produced by proteolytic cleavage, using enzymes such as papain (to produce Fab fragments) or pepsin (to produce F(ab′)2 fragments). Alternatively, secreted protein-binding fragments can be produced through the application of recombinant DNA technology or through synthetic chemistry.

An embodiment of the invention is an anti-cancer agent comprising a mammaglobin B binding protein (e.g., an anti-mammaglobin B antibody) coupled to a cytotoxic agent.

The cytotoxic agent can be a therapeutic radionuclide (e.g, Iodine-131), a toxin (e.g., diphtheria toxin), or an anti-cancer chemotherapy agent (e.g., a standard chemotherapy drug such as methotrexate). Procedures for coupling toxins, chemotherapy agents, and radionuclides to antibodies or other binding proteins are described in U.S. patent application Ser. No. 11/407,590, and international patent application PCT/US2005/37739.

The invention will now be illustrated with the following examples. The examples are intended to illustrate the invention, but not limit its scope.

### EXAMPLES

### Example 1

### Differential Gene Expression Profiles Between Tumor Biopsies and Short-Term Primary Cultures of Ovarian Carcinoma

Our group has recently used highly purified primary ovarian cancer cell lines with limited in vitro passages to investigate gene expression fingerprints of OSPC [14]. In this Example, we extended our studies by using a more extensive set of probes (Affymetrix U133A) to analyze gene expression patterns in 19 flash-frozen OSPC containing at least 70% tumor cells and compared such expression to 15 non-malignant control specimens consisting of highly purified normal ovarian surface epithelium (HOSE) short-term cultures. Finally, in order to prospectively analyze whether a short-term in vitro culture of primary OSPC (necessary to highly enrich in tumor cell OSPC extracted RNA) may significantly alter gene expression profiling results, we have also compared the genetic fingerprints of 5 highly purified primary OSPC cultures to those of 5 autologous matched flash-frozen biopsies obtained from the same individuals.

### Patients and Methods

### Ovarian Tissue Samples

Nineteen snap-frozen serous papillary ovarian carcinomas of histologically proven ovarian origin were obtained from the Division of Gynecologic Oncology at the University of Brescia, Italy. Study approval was obtained from the institutional review board, and all patients signed an informed consent according to institutional guidelines. All the ovarian cancer patients were surgically treated in the Department of Gynecologic Oncology of the University of Brescia during the last 2 years. They underwent a radical surgical tumor debulking and a complete staging procedure followed by platinum-based chemotherapy. No patients received chemotherapy before surgery. Patient clinical and pathological characteristics are showed in Table 1.

TABLE 1
Clinical and pathological characteristics of the patients

| Patient | Age | Stage | Grade | Ascites | Chemo regimen |
|---|---|---|---|---|---|
| OSPC 1 | 67 | IIIC | G3 | No | Tax + carb |
| OSPC 2 | 79 | IIIC | G3 | Yes | Carb |
| OSPC 3 | 63 | IIIC | G3 | No | Tax + carb |
| OSPC 4 | 69 | IIIC | G3 | Yes | Tax + carb |
| OSPC 5 | 24 | IV | G2 | Yes | Tax + carb |
| OSPC 6 | 74 | IIIC | G3 | Yes | Carb |
| OSPC 7 | 52 | IIIC | G3 | No | Tax + carb |
| OSPC 8 | 43 | IIIC | G3 | No | Tax + carb |
| OSPC 9 | 78 | IIB | G3 | No | Tax + carb |
| OSPC 10 | 72 | IV | G3 | Yes | Tax + carb |
| OSPC 11 | 55 | IIIC | G3 | No | Tax + carb |
| OSPC 12 | 45 | IIIC | G3 | Yes | Tax + carb |
| OSPC 13 | 45 | IV | G3 | Yes | Tax + carb |
| OSPC 14 | 61 | IIIC | G2 | Yes | Tax + carb |
| OSPC 15 | 49 | IIIC | G3 | Yes | Tax + carb |
| OSPC 16 | 43 | IIIC | G3 | Yes | Tax + carb |
| OSPC 17 | 47 | IB | G3 | No | Tax + carb |
| OSPC 18 | 84 | IIIC | G3 | No | Tax + carb |
| OSPC 19 | 71 | IIIC | G3 | Yes | Carb |

Briefly, ovarian tumor tissues were identified, sharp-dissected and snap-frozen in liquid nitrogen within 30 min from resection. The samples were embedded in O.C.T. medium, microdissected and the frozen sections were stained with H&E to check epithelial purity. Each sample was histologically analyzed by a staff pathologist, and only tumor samples containing at least 70% tumor epithelial cells were retained for further total RNA extraction.

### Establishment of OSPC and HOSE Primary Cell Lines

A total of 20 primary cell lines (5 ovarian serous papillary cancer and 15 HOSE) were established after sterile processing of samples from surgical biopsies. HOSE were derived from normal ovarian epithelial tissues of patients undergoing surgery for benign pathologies including uterine fibromas or prolapses. Pathological examination confirmed the absence of any neoplastic disease. To obtain pure HOSE short-term cell cultures, the normal ovarian tissue was macrodissected and incubated in 2 ml collagenase and DNAse for 30 min at 37° C. with occasional agitation. Sheets of HOSE cell fragments were gently scraped with a rubber scraper directly into complete growth medium M199/MCDB105 (Invitrogen/Sigma) supplemented with 10% fetal bovine serum, 200 μg/ml penicillin and 200 μg/ml streptomycin. Primary cell lines were maintained in the same complete growth medium at 37° C., 5% CO2 in tissue culture 6-well plates (Corning, N.Y.) and used to generate monolayers with no or minimal contamination (i.e., less than 1%) by other cell types as evaluated by immunocytochemical staining with antibody against pan-cytokeratin and EMA. Tumor tissue was mechanically minced to portions no larger than 1-3 mm³ in an enzyme solution made of 0.14% collagenase type I (Sigma) and 0.01% DNAse (Sigma, 2000 KU/mg) in RPMI-1640 and incubated in the same solution in a magnetic stirring apparatus for 1 h at room temperature. Enzymatically dissociated tumor cells were then washed twice in RPMI-1640 10% fetal bovine serum (FBS) and maintained in RPMI supplemented with 10% FBS, 200 μg/ml penicillin and 200 μg/ml streptomycin at 37° C., 5% CO₂ in tissue culture flasks 75 cm² or Petri dishes (Corning, N.Y.). 48-72 h after seeding on plasticware, non-adherent tumor cells and contaminant inflammatory cells were gently removed from the culture by multiple washing with phosphate-buffered saline (PBS). Tumor cultures contaminated with adherent stromal cells underwent a maximum of three rounds of gentle trypsinization to detach stromal cells before being harvested for RNA extraction. Total length of in vitro culture was less than 14 days for all samples. Normal and tumor cell cultures were collected for RNA extraction at 70-80% confluence without being subcultured (passage 0). The epithelial purity of tumor and normal ovarian cell lines was evaluated by immunocytochemical staining with antibody against pan-cytokeratin and EM as previously described [14]. Only cell cultures composed of at least 99% epithelial cells were retained for RNA extraction.

### Total RNA Extraction and Genechip Hybridization

Total RNA was obtained from a total of 39 samples including 19 flash-frozen serous papillary ovarian carcinomas, 15 HOSE and 5 ovarian serous papillary cancer primary cell lines by using TRIZOL reagent (Life technologies, Inc., Carlsbad, Calif.) and then further purified using RNeasy Min-elute Clean-up Columns (Qiagen, Valencia, Calif.) as described by the manufacturers. Tissue samples, approximately 50 mg, were previously pulverized in a chilled mortar, TRIZOL reagent was added and the tissue was homogenized for 20 seconds using Ultra-turrax T8 (IKA-WERKE).

All tissue samples' quality was strictly controlled to verify the RNA integrity before use in microarray experiments. RNA quantity was evaluated spectrophotometrically, and the quality was assessed with the Agilent 2100 bioanalyzer (Agilent technologies Inc.). Only samples with good RNA yield and no RNA degradation (28 S: 18 S>1.5 and RNA integrity>8.5) were retained for further experiments.

Labeling of samples and hybridization to the Affymetrix (Santa Clara, Calif.) Human U133A oligonucleotide microarrays chip containing 22,283 probe sets were performed following the manufacturer's protocols, as described [22].

### Data Analysis and Clustering

Gene expression values were computed using the MAS5 algorithm with target value set to 100 [23]. In order to select highly differentially expressed genes, a non-specific filtering was first applied. Genes were retained if at least 75% of samples in the overexpressed group had presence call and an expression value higher than 50. The comparison between tumor and normal samples was performed by means of the SAM algorithm [24]. Genes were considered of interest if the absolute value of the estimated fold change was equal or higher than 3, and if the q value [24] was smaller than 0.05. We also computed a posterior probability of expression, according to Efron et al. [25], whose results were consistent with the SAM analysis. A hierarchical clustering using Pearson correlation as distance matrix was performed to graphically show the results of the analysis. All the analyses were performed using the R platform [26] and the Bioconductor packages [27].

### Validation of Gene Expression by Quantitative RT-PCR

Real-time polymerase chain reaction was performed in triplicate by using primer sets and probes specific for six selected genes found to be significantly up-regulated in OSPC tissue samples compared with HOSE. These genes are: mammaglobin B, mucin 1, kallikrein 6, claudin 7, CXCR4 and mucin 16/CA125. All the reactions were carried out on the ABI PRISM 7000 Sequence detection System (Applied Biosystems, Applera UK, Cheshire, UK) using the TaqMan Universal PCR master Mix and the following Assays on Demand (Applied Biosystems): Hs00267180_m1 (mammaglobin B), Hs00159357_m1 (mucin 1), Hs00160519_m1 (kallikrein 6), Hs00600772_m1 (claudin 7), Hs00607978_s1 (CXCR4) and Hs00226715_m1 (mucin 16/CA125). In brief, complementary DNA obtained from 50 ng of total RNA was amplified in a 25-μl PCR reaction following the manufacturer's recommended protocol and amplification steps: denaturation for 10 min at 95° C. followed by 40 cycles of denaturation at 95° C. for 15 s and annealing extension at 60° C. for 1 min. The comparative threshold cycle (CT) method was used for the calculation of amplification fold as specified by the manufacturer. The housekeeping gene 18S ribosomal RNA was used to normalize the quantity of cDNA used in the PCR reactions. Spearman rank correlation was used to estimate the degree of association between microarray and q-RT-PCR data for each genes.

### Immunohistochemistry on Formalin-Fixed Tissues

To evaluate protein expression levels for some of the genes found up-regulated in OSPC compared to HOSE, immunohistochemical staining for mammaglobin 2, CXCR-4 and mucin-1 was performed on 29 samples (i.e., 19 OSPC and 10 normal ovaries), stored in the Department of Pathology at the University of Brescia, Italy. Formalin-fixed, paraffin-embedded tissue constituting the specular section of the tumor biopsies used for microarray analysis was cut and stained with H&E and analyzed by a Staff Surgical Pathologist. As controls, surface epithelia obtained from normal ovaries were used. Briefly, formalin-fixed, paraffin-embedded tissues were cut at 2 μm, mounted on charged slide and dried. For immunohistochemical analysis, slides were deparaffinized and rehydrated in graded solutions of ethanol and distilled water. Endogenous peroxidase was blocked by incubation with peroxidase blocking solution (DAKO ChemMate, CA, USA) for 15 min followed by rinsing in Tris-buffered saline (TBS). Non-specific staining was blocked by treatment with normal goat serum (1:50) for 5 min. The immunohistochemical method involved sequential amplification of primary antibody to mucin-1 (clone VU4H5, Santa Cruz Biotechnology, Inc. CA, USA; dilution 1:40), mammaglobin (mammaglobin (clone 31A5) Rabbit Monoclonal Antibody, Zeta Corporation, Sierra Madre, Calif., USA dilution 1:50) and CXCR4 (rabbit polyclonal, Novus Biologicals, Inc, dil 1:1000) for 45 min and a secondary biotinylated anti-mouse/rabbit antibody (Menarini, Florence Italy, dil 1:20) for 15 min and streptavidin-biotin complex (Reagent kit, Menarini, Florence, Italy, dil 1:20) for 15 min. The immunoprecipitate was visualized by treatment with 3′3-diaminobenzidine (Bio-optica, Milan, Italy) for 5 min and counterstained by hematoxylin (DAKO, CA, USA).

### Results

### Gene Expression Analysis and Clustering of OSPC and HOSE

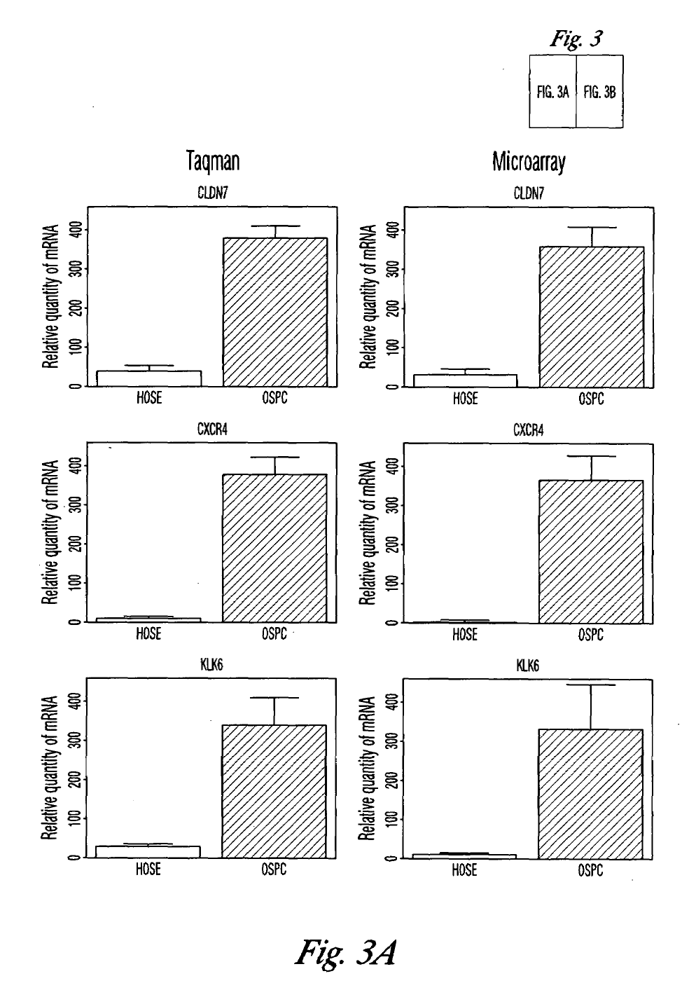

Comprehensive gene expression profiles of 19 snap-frozen OSPC and 15 HOSE cell lines were generated using high-density oligonucleotide arrays with 22,283 probe sets, which in total interrogated some 14,500 genes. Chi-square test was used to compare the homogeneity of the two groups (cases and controls) across strata defined by categories of the main clinical characteristics. The asymptotic significance was calculated using the Pearson Chi Square test. Level of significance were defined at P<0.05. No significant differences were observed in the two groups in terms of age (≧vs.<40 years), menopausal status and parity (nulli- vs. pluri-parity) (P values of 0.863, 0.336, 0.666, respectively). Using unsupervised hierarchical cluster analysis with 8637 probe sets, we identified differences in gene expression between OSPC and HOSE which readily distinguished the two groups. As shown in FIG. 2, all 19 OSPC were found to group together in the rightmost columns of the dendrogram. Similarly, in the leftmost columns, all 15 HOSE were found to cluster tightly together. After filtering out most “absent” genes, the SAM analysis by t test revealed 1458 probe sets showing >3-fold change with q value<0.05. A total of 901 genes were found significantly overexpressed in OSPC when compared to HOSE (Table 2 depicts the genes showing >15-fold change). Included in this list are SCGB2A1 (mammaglobin B), claudin 3, claudin 4, claudin 7, claudin 10, cxcr4, B7-H4, ceruloplasmin, CD24, SCNN1A, osteopontin, mucin1 and mucin16/CA125, folate receptor 1, TACSTD1 (TROP1/EpCAM) and TACSTD2 (TROP2), TNFSF10 (APO2L), WFDC2 (HE4), inhibin beta B, EBAG9, mesothelin, kallikrein 6, kallikrein 7, kallikrein 8, kallikrein 10 and kallikrein 11, CD47, erbB3, SLC34A2, S100A8, clusterin, prostasin, CDH1 (E-cadherin), TMPRSS3 (TADG12), TADG14 (neuropsin/ovasin) and ST14 (TADG15 or matriptase).

TABLE 2
Up-regulated genes expressed at least fifteen-fold higher in OSPC versus HOSE

| U133A | | Ratio | |
|---|---|---|---|
| probe set | Gene symbol | OSPC/HOSE | Gene Name |
| 205979_at | SCGB2A1 | 827.679 | Secretoglobin, family 2A, member 1 |
| | | | (mammaglobin B) |
| 203953_s_at | CLDN3 | 241.667 | Claudin 3 |
| 211430_s_at | IGHG3 | 239.465 | Immunoglobulin heavy constant gamma 3 |
| 214677_x_at | IGLJ3 | 194.061 | Immunoglobulin lambda joining 3 |
| 216834_at | RGS1 | 156.809 | Regulator of G-protein signaling 1 |
| 213975_s_at | LYZ | 127.682 | Lysozyme (renal amyloidosis) |
| 210982_s_at | HLA-DRA | 127.33 | Major histocompatibility complex, class II, DR |
| | | | alpha |
| 208498_s_at | AMY1A | 119.133 | Amylase, alpha 1A |
| 201839_s_at | TACSTD1 | 99.478 | Tumor-associated calcium signal transducer 1 |
| 213994_s_at | SPON1 | 97.324 | Spondin 1, extracellular matrix protein |
| 221671_x_at | IGKC | 87.698 | Immunoglobulin kappa variable 1–5 |
| 217028_at | CXCR4 | 86.754 | Chemokine (C—X—C motif) receptor 4 |
| 204439_at | C1orf29 | 78.101 | Interferon-induced protein 44 |
| 219768_at | B7-H4 | 76.458 | Immune costimulatory protein B7-H4 |
| 204846_at | CP | 71.745 | Ceruloplasmin (ferroxidase) |
| 202489_s_at | FXYD3 | 71.579 | FXYD domain containing ion transport regulator 3 |
| 206799_at | SCGB1D2 | 71.185 | Secretoglobin, family 1D, member 2 |
| 221884_at | EVI1 | 70.713 | Ecotropic viral integration site 1 |
| 266_s_at | CD24 | 69.719 | CD24 (small cell lung carcinoma cluster 4 |
| | | | antigen) |
| 219274_at | TM4SF12 | 68.782 | Transmembrane 4 superfamily member 12 |
| 209875_s_at | SPP1 | 63.499 | Secreted phosphoprotein 1 (osteopontin) |
| 221651_x_at | IGKC | 62.305 | Immunoglobulin kappa variable 1–5 |
| 203954_x_at | CLDN3 | 61.306 | Claudin 3 |
| 203780_at | EVA1 | 57.903 | Epithelial V-like antigen 1 |
| 209074_s_at | TU3A | 57.811 | TU3A protein |
| 209138_x_at | NA | 57.274 | Immunoglobulin lambda joining 3 |
| 212671_s_at | HLA-DQA1 | 54.333 | Major histocompatibility complex, class II, DQ |
| 218232_at | C1QA | 47.92 | Complement component 1 |
| 209771_x_at | CD24 | 47.648 | CD24 (small cell lung carcinoma cluster 4 |
| | | | antigen) |
| 216379_x_at | KIAA1919 | 45.107 | KIAA1919 |
| 209772_s_at | CD24 | 44.299 | CD24 (small cell lung carcinoma cluster 4 |
| | | | antigen) |
| 219607_s_at | MS4A4A | 43.486 | Membrane-spanning 4-domains, subfamily A, 4 |
| 212560_at | SORL1 | 41.117 | Sortilin-related receptor, L (DLR class) A |
| | | | repeats |
| 203453_at | SCNN1A | 40.846 | Sodium channel, nonvoltage-gated 1 alpha |
| 209201_x_at | CXCR4 | 40.106 | Chemokine (C—X—C motif) receptor 4 |
| 215121_x_at | NA | 39.405 | Immunoglobulin lambda locus |
| 213693_s_at | MUC1 | 39.114 | Mucin 1, transmembrane |
| 222281_s_at | NA | 38.738 | NA |
| 219850_s_at | EHF | 35.058 | Ets homologous factor |
| 215049_x_at | CD163 | 35.021 | CD163 antigen |
| 214669_x_at | IGKV3D-15 | 34.433 | Immunoglobulin kappa variable 3D-15 |
| 208650_s_at | CD24 | 33.589 | CD24 (small cell lung carcinoma cluster 4 |
| | | | antigen) |
| 205473_at | ATP6V1B1 | 33.568 | ATPase, H+ transporting, lysosomal 56/58 kDa |
| 203645_s_at | CD163 | 32.567 | CD163 antigen |
| 204437_s_at | FOLR1 | 31.182 | Folate receptor 1 (adult) |
| 219121_s_at | FLJ20171 | 31.116 | Hypothetical protein FLJ20171 |
| 202286_s_at | TACSTD2 | 31.016 | Tumor-associated calcium signal transducer 2 |
| 204416_x_at | APOC1 | 30.921 | Apolipoprotein C-I |
| 211699_x_at | HBA1 | 29.63 | Hemoglobin, alpha 1 |
| 213993_at | SPON1 | 29.017 | Spondin 1, extracellular matrix protein |
| 204122_at | TYROBP | 28.072 | TYRO protein tyrosine kinase binding protein |
| 212998_x_at | HLA-DQB1 | 27.58 | Major histocompatibility complex, class II, DQ |
| | | | beta 1 |
| 202953_at | C1QB | 26.524 | Complement component 1 |
| 200795_at | SPARCL1 | 25.349 | SPARC-like 1 (mast9, hevin) |
| 210397_at | DEFB1 | 25.085 | Defensin, beta 1 |
| 212588_at | PTPRC | 24.647 | Protein tyrosine phosphatase, receptor type, C |
| 205898_at | CX3CR1 | 24.22 | Chemokine (C—X3—C motif) receptor 1 |
| 219993_at | SOX17 | 24.05 | SRY (sex determining region Y)-box 17 |
| 208894_at | HLA-DRA | 23.663 | Major histocompatibility complex, class II, DR |
| 208651_x_at | CD24 | 23.223 | CD24 (small cell lung carcinoma cluster 4 |
| | | | antigen) |
| 210387_at | HIST1H2BG | 21.33 | Histone 1, H2bg |
| 202688_at | TNFSF10 | 21.325 | Tumor necrosis factor (ligand) superfamily, 10 |
| 201720_s_at | LAPTMS | 21.27 | Lysosomal associated multispanning membrane |
| | | | protein 5 |
| 205569_at | LAMP3 | 21.116 | Lysosomal-associated membrane protein 3 |
| 201721_s_at | LAPTM5 | 21.105 | Lysosomal associated multispanning membrane |
| | | | protein 5 |
| 218723_s_at | RGC32 | 20.546 | Response gene to complement 32 |
| 201428_at | CLDN4 | 20.199 | Claudin 4 |
| 205225_at | ESR1 | 19.033 | Estrogen receptor 1 |
| 214414_x_at | HBA1 | 18.631 | Hemoglobin, alpha 1 |
| 204039_at | CEBPA | 18.61 | CCAAT/enhancer binding protein (C/EBP), |
| | | | alpha |
| 204006_s_at | FCGR3B | 17.75 | Fc fragment of IgG, low affinity IIIb, CD16 |
| 212188_at | KCTD12 | 17.653 | Potassium channel tetramerization domain 12 |
| 218963_s_at | KRT23 | 17.616 | Keratin 23 (histone deacetylase inducible) |
| 209436_at | SPON1 | 17.533 | Spondin 1, extracellular matrix protein |
| 208998_at | UCP2 | 17.37 | Uncoupling protein 2 (mitochondrial, proton |
| | | | carrier) |
| 211991_s_at | HLA-DPA1 | 17.136 | Major histocompatibility complex, class II, DP |
| 219961_s_at | C20orf19 | 17.097 | Chromosome 20 open reading frame 19 |
| 208981_at | PECAM1 | 16.912 | Platelet/endothelial cell adhesion molecule |
| | | | (CD31) |
| 202790_at | CLDN7 | 16.289 | Claudin 7 |
| 205350_at | CRABP1 | 16.045 | Cellular retinoic acid binding protein 1 |
| 202917_s_at | S100A8 | 15.765 | S100 calcium binding protein A8 (calgranulin A) |
| 211429_s_at | SERPINA1 | 15.742 | c-myc promoter binding protein |
| 212943_at | KIAA0528 | 15.727 | KIAA0528 gene product |
| 202833_s_at | SERPINA1 | 15.717 | Serine (or cysteine) proteinase inhibitor, clade A |
| 202687_s_at | TNFSF10 | 15.586 | Tumor necrosis factor (ligand) superfamily, 10 |
| 202800_at | SLC1A3 | 15.537 | Solute carrier family 1, member 3 |
| 203186_s_at | S100A4 | 15.313 | S100 calcium binding protein A4 |
| 204533_at | CXCL10 | 15.283 | Chemokine (C—X—C motif) ligand 10 |
| 206385_s_at | ANK3 | 15.27 | Ankyrin 3, node of Ranvier (ankyrin G) |
| 207847_s_at | MUC1 | 15.219 | Mucin 1, transmembrane |
| 218186_at | RAB25 | 15.048 | RAB25, member RAS oncogene family |

The second profile was represented by 557 genes underexpressed in OSPC and overexpressed in HOSE. This list includes natriuretic peptide precursor B, plasminogen activator inhibitor type 1, aldehyde dehydrogenase 1 member A3, calbindin 2, lysyl oxidase, dual specific phosphatase 1, neuropeptide Y, vitronectin to mention just a few (Table 3 depicts the genes showing >15-fold change).

TABLE 3
Up-regulated genes expressed at least fifteen folds higher in HOSE versus OSPC

| U133A | | Ratio | |
|---|---|---|---|
| probe set | Gene symbol | HOSE/OSPC | Gene name |
| 206801_at | NPPB | 404.21832 | Natriuretic peptide precursor B |
| 202628_s_at | SERPINE1 | 178.30845 | Serine (or cysteine) proteinase inhibitor |
| 203180_at | ALDH1A3 | 113.07810 | Aldehyde dehydrogenase 1 family, member A3 |
| 204338_s_at | RGS4 | 107.78327 | Regulator of G-protein signaling 4 |
| 202627_s_at | SERPINE1 | 77.345659 | Serine (or cysteine) proteinase inhibitor |
| 210702_s_at | PTGIS | 70.708583 | Prostaglandin I2 (prostacyclin) synthase |
| 205428_s_at | CALB2 | 69.424427 | Calbindin 2, 29 kDa (calretinin) |
| 208606_s_at | WNT4 | 62.479519 | Wingless-type MMTV integration site family, |
| | | | member 4 |
| 205502_at | CYP17A1 | 58.968195 | Cytochrome P450, family 17, subfamily A, |
| | | | polypeptide 1 |
| 203074_at | ANXA8 | 52.368445 | Annexin A8 |
| 204548_at | STAR | 50.5558865 | Steroidogenic acute regulator |
| 213640_s_at | LOX | 48.318218 | Lysyl oxidase |
| 204879_at | T1A-2 | 47.447780 | Lung type-I cell membrane-associated |
| | | | glycoprotein |
| 206336_at | CXCL6 | 46.956240 | Chemokine (C—X—C motif) ligand 6 |
| 204298_s_at | LOX | 45.800364 | Lysyl oxidase |
| 208131_s_at | PTGIS | 44.654935 | Prostaglandin I2 (prostacyclin) synthase |
| 207510_at | BDKRB1 | 42.143520 | Bradykinin receptor B1 |
| 208539_x_at | SPRR2B | 36.320932 | Small proline-rich protein 2B |
| 201044_x_at | DUSP1 | 34.002829 | Dual specificity phosphatase 1 |
| 203951_at | CNN1 | 32.1488973 | Calponin 1, basic, smooth muscle |
| 221870_at | EHD2 | 30.674316 | EH-domain containing 2 |
| 204515_at | HSD3B1 | 30.39021 | Hydroxy-delta-5-steroid dehydrogenase, 3 beta |
| 207016_s_at | ALDH1A2 | 28.546029 | Aldehyde dehydrogenase 1 family, member A2 |
| 204614_at | SERPINB2 | 28.389305 | Serine (or cysteine) proteinase inhibitor |
| 215446_s_at | LOX | 27.456398 | Lysyl oxidase |
| 206001_at | NPY | 26.439371 | Neuropeptide Y |
| 213112_s_at | SQSTM1 | 24.110188 | Sequestosome 1 |
| 211892_s_at | PTGIS | 23.647227 | Prostaglandin I2 (prostacyclin) synthase |
| 204602_at | DKK1 | 21.267390 | Dickkopf homolog 1 (Xenopus laevis) |
| 204534_at | VTN | 21.239959 | Vitronectin (somatomedin B, complement S- |
| | | | protein) |
| 219263_at | RNF128 | 20.595412 | Ring finger protein 128 |
| 205289_at | BMP2 | 20.568487 | Bone morphogenetic protein 2 |
| 203695_s_at | DFNA5 | 20.4080567 | Deafness, autosomal dominant 5 |
| 203886_s_at | FBLN2 | 20.0862035 | Fibulin 2 |
| 204627_s_at | ITGB3 | 19.453156 | Integrin, beta 3 (platelet glycoprotein IIIa, |
| | | | antigen CD61) |
| 207826_s_at | ID3 | 19.445737 | Inhibitor of DNA binding 3 |
| 203851_at | IGFBP6 | 18.533424 | Insulin-like growth factor binding protein 6 |
| 202274_at | ACTG2 | 18.234359 | Actin, gamma 2, smooth muscle, enteric |
| 207876_s_at | FLNC | 17.991080 | Filamin C, gamma (actin binding protein 280) |
| 201645_at | TNC | 17.865338 | Tenascin C (hexabrachion) |
| 203304_at | BAMBI | 17.562034 | BMP and activin membrane-bound inhibitor |
| | | | homolog |
| 218934_s_at | HSPB7 | 17.294098 | Heat shock 27 kDa protein family, member 7 |
| 203060_s_at | PAPSS2 | 16.730008 | 3′-phosphoadenosine 5′-phosphosulfate synthase 2 |
| 220091_at | SLC2A6 | 16.685717 | Solute carrier family 2, member 6 |
| 208621_s_at | VIL2 | 15.669842 | Villin 2 (ezrin) |
| 205832_at | CPA4 | 15.511641 | Carboxypeptidase A4 |
| 204540_at | EEF1A2 | 15.1671159 | Eukaryotic translation elongation factor 1 alpha 2 |
| 217234_s_at | VIL2 | 15.1443332 | Villin 2 (ezrin) |
| 204420_at | DIPA | 15.093103 | FOS-like antigen 1 |
| 205207_at | IL6 | 15.038225 | Interleukin 6 (interferon, beta 2) |
| 204802_at | RRAD | 15.003273 | Ras-related associated with diabetes |

To analyze whether gene expression profiling obtained comparing snap-frozen ovarian cancer tissues to HOSE may significantly differ from those obtained from highly purify short-term culture of ovarian tumor cells, we performed gene expression profiling to compare the genetic fingerprints of 5 snap-frozen ovarian cancer tissues to those obtained from the matched OSPC short-term cultures. SAM analysis for paired samples involving 8637 genes found only 31 probe sets (0.35%) differentially expressed between snap-frozen tissue OSPC and short-term OSPC cultures (Table 4). Most of the differentially expressed probe sets (27 out or 31) were found to be up-regulated in the snap-frozen OSPC tissues when compared to short-term cultures and to encode genes characterizing immune system cells and/or extracellular matrix components. In contrast, three genes were found up-regulated in the short-term cultures when compared to snap-frozen OSPC. These genes are interleukin 6, COPA and villin 2 (Table 4).

TABLE 4
List of genes differentially expressed between flash-frozen OSPC
biopsies and primary OSPC short-term cultures

| U133A | | Ratio | |
|---|---|---|---|
| probe set | Gene symbol | biopsy/primary | Gene name |
| 211699_x_at | HBA1 | 178.41 | Hemoglobin, alpha 1 |
| 209116_x_at | HBB | 67.794 | Hemoglobin, beta |
| 214414_x_at | HBA1 | 65.953 | Hemoglobin, alpha 1 |
| 211745_x_at | HBA2 | 61.127 | Hemoglobin, alpha 2 |
| 204018_x_at | HBA1 | 58.593 | Hemoglobin, alpha 1 |
| 200795_at | SPARCL1 | 54.981 | SPARC-like 1 (mast9, hevin) |
| 216834_at | RGS1 | 35.876 | Regulator of G-protein signaling |
| 211696_x_at | HBB | 26.967 | Hemoglobin, beta |
| 205979_at | SCGB2A1 | 21.234 | Secretoglobin, family 2°, member 1 |
| 209541_at | IGF1 | 19.052 | Insulin-like growth factor 1 |
| 217232_x_at | HBB | 18.903 | Hemoglobin, beta |
| 221558_s_at | LEF1 | 14.499 | Lymphoid enhancer-binding factor-1 |
| 205350_at | CRABP1 | 10.824 | Cellular retinoic acid binding protein 1 |
| 215049_x_at | CD163 | 10.516 | CD163 antigen |
| 219666_at | MS4A6A | 9.7954 | Membrane-spanning 4-domains, subfamily A |
| 214428_x_at | C4A | 9.73 | Complement component 4A |
| 218736_s_at | PALMD | 8.748 | Palmdelphin |
| 202953_at | C1QB | 8.361 | Complement component 1, |
| 202878_s_at | C1QR1 | 8.337 | Complement component 1, q receptor 1 |
| 212588_at | PTPRC | 7.471 | Protein tyrosine phosphatase, R type C |
| 208982_at | PECAM1 | 7.084 | Platelet/endothelial cell adhesion molecule |
| | | | (CD31) |
| 221763_at | JMJD1C | 5.268 | Jumonji domain containing 1C |
| 204249_s_at | LMO2 | 5.242 | LIM domain only 2 (rhombotin-like 1) |
| 204061_at | PRKX | 5.129 | Protein kinase, X-linked |
| 214433_s_at | SELENBP1 | 4.702 | Selenium binding protein 1 |
| 214375_at | PPFIBP1 | 4.501 | PTPRF interacting protein, binding protein 1 |
| 202917_s_at | S100A8 | 3.942 | S100 calcium binding protein A8 (calgranulin A) |
| 214336_s_at | COPA | 0.21 | Coatomer protein complex, subunit alpha |
| 217234_s_at | VIL2 | 0.117 | Villin 2 (ezrin) |
| 208621_s_at | VIL2 | 0.111 | Villin 2 (ezrin) |
| 205207_at | IL6 | 0.084 | Interleukin 6 |

### Validation of Gene Expression by Quantitative RT-PCR

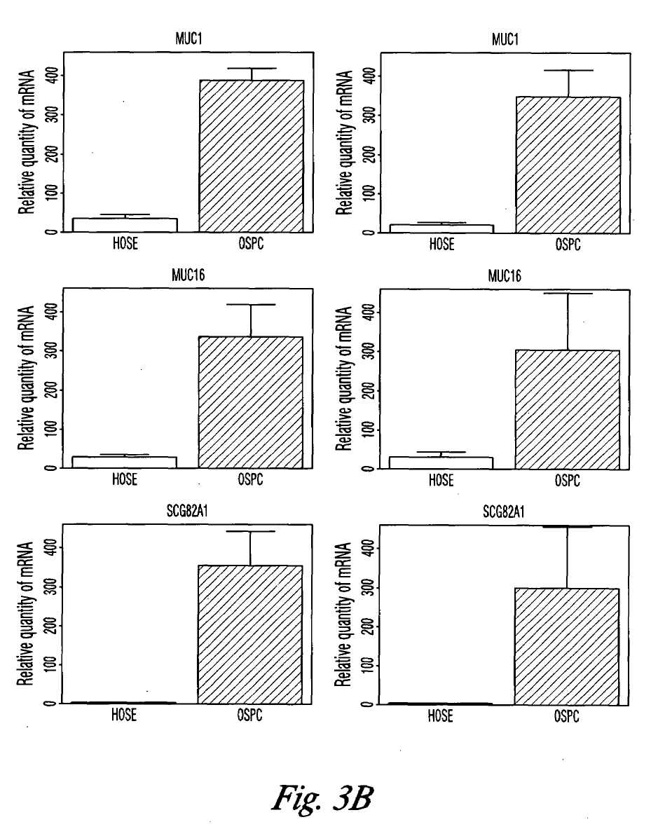

Six highly differentially expressed genes between OSPC and HOSE (i.e., CLDN7, CXR4, KLK6, MUC1, SCGB2A1 and MUC16/CA125) were selected for q-RT-PCR analysis. A comparison of the microarray and q-RT-PCR data for these genes is shown in FIG. 3. For all six genes tested, the quantitative real-time PCR data were highly correlated to the microarray data (P<0.001; rs CLDN7=0.88, rs CXR4=0.92, rs KLK6=0.95, rs MUC1=0.92, rs SCGB2A1=0.86, rs MUC16=0.86), as estimated from the samples (i.e., 19 OSPC and 15 HOSE) included in both the q-RT-PCR and microarray experiments. Thus, q-RT-PCR data suggest that most array probe sets are likely to accurately measure the levels of the intended transcript within a complex mixture of transcripts.

### Validation of Protein Expression by Immunohistochemical Staining

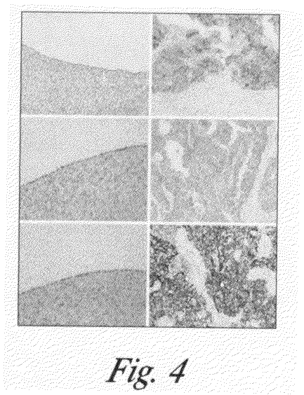

To confirm gene expression results at the protein level, IHC for mammaglobin-2, CXCR4 and mucin 1 was carried out on 19 formalin-fixed tumor and 10 normal samples. As representatively shown in the left panel of FIG. 4, a strong membranous and cytoplasmic staining for CXCR4 and mucin 1 was detected in all 19 OSPC samples tested, while no expression was found in normal ovarian epithelium. Similarly, a high cytoplasmic staining for mammaglobin-2 in more than 60% of tumor cells was detected in all 19 OSPC samples tested, while no expression was found in normal ovarian epithelium (FIG. 4).

### Discussion

With the goal to identify genes potentially useful as novel diagnostic and/or therapeutic markers for ovarian cancer, in this study, we evaluated the gene expression profiles of 19 ovarian serous papillary flash-frozen carcinomas and compared their genetic fingerprints to those of 15 HOSE short-term cultures using oligonucleotide microarrays complementary to >14,500 human genes. In addition, because our previous studies evaluating the genetic fingerprints of ovarian carcinoma involved the use of highly purified primary OSPC cultures and recent reports have raised concerns about the degree to which cultured cells may reflect their cell of origin [14] and [21], in this study, we have also performed a direct comparison of the gene expression profiling of flash-frozen ovarian cancer tissue biopsies to those derived from short-term primary ovarian cancer cell lines derived from matched ovarian specimens.

We found that hierarchical clustering of the samples and gene expression levels within the samples led to the unambiguous separation of OSPC and HOSE. After filtering out most ‘absent’ genes, the SAM analysis by t test revealed 1458 probe sets showing >3-fold change with q value<0.05. A total of 901 probe sets were found significantly overexpressed in OSPC when compared to HOSE while 557 probe sets were found up-regulated in HOSE when compared to OSPC. Several of the genes found highly differentially expressed in our series of flash-frozen OSPC samples have been previously identified by our group as well as others as highly expressed in OSPC when compared to HOSE, while many others represent novel findings. These results obtained using a more extensive set of probes (i.e., 22,283 versus 15,833) and flash-frozen tissue instead of primary cultures validate further our experimental approach as well as our criteria to determine the genes differentially expressed. Of interest, many of the genes up-regulated in flash-frozen ovarian cancer were found to represent surface or secreted proteins such as mammaglobin 2, laminin, claudin 3 and claudin 4, B7-H4, tumor-associated calcium signal transducer 1 and 2 (TROP-1/Ep-CAM; TROP-2), ladinin 1, S100A2, SERPIN2 (PAI-2), CD24, lipocalin 2, osteopontin, kallikrein 6 (protease M), kallikrein 7 and kallikrein 10, matriptase (TADG-15) and stratifin. The known function of some of these genes may provide insights in the biology of serous ovarian tumors, while others may prove to be useful diagnostic and therapeutic markers against OSPC.

For instance, mammaglobin 2 (SCGB2A1, also known as mammaglobin B, mammaglobin-2, and MGB-2) was found as the top differentially expressed gene in OSPC when compared to HOSE (827-fold). SCGB2A1 is a gene originally isolated in rat uterus and human endometrium [28] and [29], encoding for a 95-amino-acid secreted protein of 10 kDa. SCGB2A1 has been previously reported overexpressed in breast, uterus and salivary glands [28] and [29] and shares very high homology with mammaglobin A, a promising serum biomarker for breast carcinoma [30], [31] and [32]. Of interest, in our study, SCGB2A1 was detected at high levels in 100% (19 out of 19) of the ovarian cancers tested, while its expression was not detectable in any of the 15 HOSE controls. In this regard, it is worth mentioning that MUC16/CA125, the best characterized and clinically useful marker for epithelial ovarian cancer, was detected in 84% (16 out 19) of our OSPC samples. Of further interest, SCGB2A1 was also highly expressed in both the flash-frozen tumor tissue collected from the two early-staged ovarian cancer patients (IB, IIB) present in our series. Finally, SCGB2A1 expression at RNA and protein levels was validated successfully in all 19 tumor samples analyzed by quantitative RT-PCR and immunohistochemistry. These data highlight the use of SCGB2A1 as a novel screening marker for ovarian cancer patients. Strikingly, however, the SCGB2A1 gene was among the few genes (i.e., less than 0.35% of the total analyzed) whose expression is rapidly down-regulated after few days of in vitro culture. Although the reason why SCGB2A1 gene expression is quickly decreased in vitro remains poorly understood, this finding may explain why in our previous report analyzing OSPC genetic fingerprints in highly purified primary ovarian cancer cell lines SCGB2A1 was not identified as one of the top highly differentially expressed genes [14].

Kallikreins belong to a family of serine proteases endowed with well-characterized roles in diverse cellular activities including blood coagulation, wound healing, digestion and immune responses, as well as tumor invasion and metastasis [33]. In this study, we found that a large number of kallikreins including human kallikrein 6, 7, 8, 10 and 11 were highly differentially expressed in OSPC when compared to HOSE. Because kallikreins are secreted proteins, and some of these family members have already found important clinical application as prostate cancer biomarkers (i.e., prostate-specific antigen, PSA) [33] and [34], these enzymes may represent useful diagnostic markers for the clinical monitoring of ovarian cancer, particularly in patients whose ovarian cancer does not express the CA125 marker [35] and [36]. Consistent with this view, multiple kallikrein gene family members, including the enzymes hK4, hK5, hK6, hK7, hK8 and hK9, have recently been shown to have prognostic significance in this disease (reviewed in [33] and [34]). Taken together, our data further support the notion that kallikreins may represent promising novel biomarkers for early detection of recurrent OSPC disease and/or for the monitoring of OSPC response to adjuvant therapy. Furthermore, because serine proteases are involved in cancer progression, they may be suitable candidates not only for diagnostic and prognostic purposes but also as novel therapeutic targets. In agreement with this hypothesis, we have recently reported the definition of an immunogenic region within kallikrein 7 (also known as stratum corneum chymotryptic enzyme, SCCE) which incorporates multiple CD8+ cytotoxic T lymphocytes epitopes as well as CD4+ T helper epitopes [37] and [38]. This discovery has highlighted the potential use of serine proteases as attractive target antigens for the immunotherapy of human ovarian cancer patients refractory to standard treatment modalities [37] and [38].

Claudin 3, claudin 4 and claudin 7, which are members of a family of tight junction proteins, were found as top differentially expressed genes in this study. These results are consistent with our previous report using primary tumor cultures [14] as well as those of others on ovarian cancer gene expression profiling using flash-frozen tumor tissues [6] and [9]. Importantly, although the exact function of claudin 3 and claudin 4 overexpression in ovarian cancer is still unclear, these proteins have recently been shown to represent the natural receptors for Clostridium perfringens enterotoxin (CPE) and to be the only family members of the transmembrane tissue-specific claudin proteins capable of mediating CPE binding and cytolysis [39]. Because CPE triggers lysis of epithelial cells through interaction with claudin 3 and claudin 4, with resultant initiation of massive permeability changes, osmotic cell ballooning and lysis within a few minutes, while mammalian cells that do not express either claudin 3 and/or claudin 4 fail to bind CPE and are not susceptible to CPE cytotoxicity [39], CPE-mediated therapy may represent a novel, potentially highly effective strategy for the treatment of ovarian cancer resistant to chemotherapy as well as other biologically aggressive human tumors overexpressing claudin 3 and/or claudin 4 [40], [41], [42] and [43]. Consistent with this hypothesis, we have recently reported the effect of exposure of primary ovarian cancer overexpressing claudin 3 and/or claudin 4 to recombinant CPE in vitro [43]. We found that, regardless of the resistance to chemotherapy of these tumors, exposure to 3.3 μg/ml of CPE may rapidly kill the primary tumors in vitro. More importantly, when the in vivo efficacy of intraperitoneal (i.p.) CPE therapy was tested in SCID mouse xenografts of chemotherapy-resistant freshly explanted human ovarian cancer, multiple i.p. administration of sublethal doses of CPE significantly inhibited tumor growth in 100% of mice harboring 1 week established ovarian tumors while repeated i.p. doses of CPE had a significant inhibitory effect on tumor progression with extended survival of animals harboring large ovarian tumor burdens (i.e., 4 weeks established). These findings, combined with the consistent high expression of claudin 3 and claudin 4 confirmed in this study, further suggest that CPE may have potential as a novel treatment for chemotherapy-resistant/recurrent ovarian cancer.

The B7 protein family provides both potent stimulatory and inhibitory regulation of T cell responses, depending on which B7 ligand and receptor are engaged on the target cell [44] and [45]. B7-H4 is a recently discovered member of the B7 family playing a major role as negative regulator of T cell responses in vitro by inhibiting proliferation, cell-cycle progression and cytokine production of CD4+ and CD8+ T cells [46], [47] and [48]. Importantly, overexpression of B7-H4 in human ovarian tumors has been recently reported to correlate with increase tumor formation in SCID mice and to promote epithelial cell transformation in ovarian cancer [49]. Consistent with this view, in our study, B7-H4 was detected at high levels in 100% (19 out of 19) of the ovarian cancers tested, while its expression was not detectable in any of the 15 HOSE controls. These data are therefore consistent with a highly restricted normal tissue distribution of B7-H4, and taken all together, these results support the use of B7-H4 as a new target for therapeutic intervention in human ovarian cancer.

Other highly ranked genes in OSPC included MUC-1, CXCR-4, laminin, tumor-associated calcium signal transducer 1 and 2 (TROP-1/Ep-CAM; TROP-2), ladinin 1, S100A2, SERPIN2 (PAI-2), CD24, lipocalin 2, osteopontin and stratifin.

Finally, when the gene expression profiling of flash-frozen ovarian cancer tissue biopsies was compared to those of short-term primary ovarian cancer cell lines derived from matched ovarian specimens, we found that only 31 out of 8637 probe sets (i.e., 0.35%) were significantly differentially expressed between the two groups. Most of the probe sets up-regulated in the flash-frozen ovarian cancer fingerprints when compared to those derived from short-term cultures were found to represent genes known to be highly expressed in non-epithelial cell types (i.e., stromal cells and immune system cells, Table 4). In contrast, only three genes we found up-regulated in the primary tumor cultures when compared to flash-frozen tissue. These genes were found to encode for either stress response proteins (i.e., interleukin 6), adhesion molecules (VIL2) or involved in the intracellular transport of vesicles (COPA). Taken all together, these data suggest that a short-term in vitro culture of primary OSPC specimens, which is necessary to highly enrich the percentage of tumor cells for RNA extraction (i.e., all samples were above 99% purity in this study) may not significantly affect ovarian tumor gene expression profiling when compared to those of flash-frozen matched samples. These results are in strong contrast with the major divergence in gene expression reported in our previous report when established serous papillary ovarian carcinoma cell lines were compared to primary ovarian tumors [14].

A large number of down-regulated genes in OSPC versus HOSE have been found in our analysis. Some of these genes encode for widely held tumor suppressor genes, such as ARH1 and Dab2/DOC2 [50], others for proteins important for ovarian tissue homeostasis or that have been previously implicated in apoptosis, proliferation, adhesion or tissue maintenance. Because of space limitations, we will not comment further upon the cluster of genes that showed down-regulation of the transcripts in invasive tumors.

In conclusion, our statistical analysis has identified a significant number of genes strongly and consistently differentially expressed in OSPC when compared to normal HOSE. We have also reported, to our knowledge for the first time, that highly purified primary ovarian tumors cultured for a limited number of passages in vitro express genetic fingerprints very similar to those of unmanipulated tumor biopsies obtained from matched tumor samples. The current investigation of several of these genes as novel diagnostic and/or therapeutic markers in ovarian carcinoma patients is expected to facilitate progress in understanding the etiology of this disease as well as its improvement in clinical management.

### Example 2

### Overexpression of Mammaglobin B in Epithelial Ovarian Carcinomas

### Patients and Methods

A total of 137 patients treated at the Division of Gynecologic Oncology of the University of Brescia, Italy, were enrolled in the study. Patient characteristics are described in Table 5. All patients diagnosed with epithelial ovarian carcinoma (EOC) underwent radical surgical tumor debulking at the University of Brescia in the last 3 years. No patient received chemotherapy before the collection of tumor biopsy. Of the 97 EOC included in the study, 61 were primary ovarian carcinomas, 28 were serous papillary omental mestases, and 8 were borderline ovarian tumors. Histologic types of EOC evaluated included 48 serous papillary tumors, 17 endometrioid, 8 clear-cell, 8 mixed, 5 undifferentiated, and 3 mucinous. Twenty-six women diagnosed with benign ovarian tumors and 14 patients with normal ovaries undergoing oopherectomy for uterine fibromas or prolapse were also enrolled in the study.

TABLE 5
Clinical and pathological characteristics of patients.

| | | Number of | Age | | | Grading | | | | Stage | | | | |
|---|---|---|---|---|---|---|---|---|---|---|---|---|---|---|
| Pathology | Histotype | Patients | Range (Y) | Mean (Y) | SD | 1 | 2 | 3 | NA | I | II | III | IV | NA |
| None | Normal | 14 | 28–79 | 54 | 12 | | | | | | | | | |
| | Ovary | | | | | | | | | | | | | |
| Benign | Serous | 14 | 21–89 | 54 | 18 | | | | | | | | | |
| | cystadenoma | | | | | | | | | | | | | |
| | Mucinous | 12 | 26–79 | 50 | 17 | | | | | | | | | |
| | cystadenoma | | | | | | | | | | | | | |
| Primary | Serous- | 20 | 24–79 | 60 | 16 | | 2 | 17 | 1 | 1 | 1 | 14 | 3 | 1 |
| EOC | papillary | | | | | | | | | | | | | |
| | Endometrioid | 17 | 34–72 | 58 | 11 | 4 | 7 | 6 | | 7 | 4 | 5 | 1 | |
| | Clear-cell | 8 | 34–84 | 59 | 17 | | | 8 | | 2 | 4 | 2 | | |
| | Mucinous | 3 | 42–85 | 61 | 18 | 2 | | 1 | | 2 | | 1 | | |
| | Mixed | 8 | 36–89 | 67 | 16 | 1 | 1 | 6 | | 1 | | 7 | | |
| | Undifferentiated | 5 | 38–84 | 61 | 17 | | | 5 | | | 1 | 3 | 1 | |
| | Borderline | 8 | 42–84 | 64 | 15 | | | | | 5 | | 2 | | 1 |
| Omental | Serous- | 28 | 24–84 | 62 | 15 | | 3 | 25 | | | | 23 | 5 | |
| Metastases | papillary | | | | | | | | | | | | | |

### Ovarian Tissue Samples

Study approval was obtained from the Institutional Review Board and all patients signed an informed consent according to institutional guidelines. Briefly, tumor tissues were identified, sharp-dissected and snap-frozen in liquid nitrogen within 30 minutes from resection. The samples were embedded in O.C.T. medium, microdisected and the frozen sections were stained with hematoxylin and eosin (HE) to check epithelial purity. Each sample was histologically analyzed by a staff pathologist and only tumor samples containing at least 70% tumor epithelial cells were retained for further total RNA extraction.

### Establishment of HOSE Primary Cell Lines

A total of 14 human ovarian surface epithelium (HOSE) primary cell lines were established after sterile processing of samples from surgical biopsies. HOSE were derived from normal ovarian epithelial tissues of patients undergoing surgery for benign pathologies. Pathological examination confirmed the absence of any neoplastic disease. To obtain pure HOSE short term cultures, the normal ovarian tissue was macrodissected and incubated in 2 ml collagenase and DNAse for 30 minutes at 37° C., 5% CO₂ in tissue culture 6-well plates (Corning, N.Y.) and used to generate monolayers. Total length of in vitro culture was less than 14 days for all samples. Normal cell cultures were collected for RNA extraction at 70%-80% confluence without being subcultured (passage 0). The epithelial purity of normal ovarian cell lines was evaluated by immunocytochemical staining with antibody against pancytokeratin and epithelial membrane antigen (EMA) as previously described [59]. Only cell cultures composed of at least 99% epithelial cells were retained for RNA extraction.

### Total RNA Extraction and Reverse Transcription

Total RNA was obtained from 82 samples including 53 primary ovarian cancer tissues with different histologies, 15 omental metastases and 14 HOSE primary cell lines (Table 6). Thirty μg of frozen tissue were sharply dissected from each sample and homogenized with a rotary homogenizer (QIAGEN, Valencia, Calif., USA) in RNeasy lysis buffer (QIAGEN, Valencia, Calif., USA). Total RNA was prepared from tissues and cells using the RNeasy Mini kit (QIAGEN, Valencia, Calif., USA). TaqMan Gene Expression Assays used in this study span an exon-exon junction eliminating the possibility of amplifying genomic DNA. Purity and RNA quantity were evaluated spectrophotometrically. The RNA integrity was tested on Agilent 2100 Bioanalyser and only RNA samples having an OD 260/280 ratio>1.8 and an Integrity Number>8.5 were retained for further amplification. For the generation of first-strand cDNA, 1 μg of total RNA was reverse-transcribed using random hexamers in a final volume of 20 μl according to the SuperScript™ II RT RNaseH-Reverse Transcriptase protocol (Invitrogen Life Technologies, Carlsbad, Calif., USA).

### Quantitative-RealTime-PCR

Real-time polymerase chain reaction was performed in duplicate by using primer set and probe specific for hMAM-B gene. All the reactions were carried out on the ABI PRISM 7000 Sequence detection System (Applied Biosystems, Applera UK, Cheshire, UK) using the TaqMan Universal PCR master Mix and the following Assay on Demand (Applied Biosystems, Applera UK, Cheshire, UK): Hs00267180_m1 (Mammaglobin B) and Hs 99999905_m1 (GAPDH). Five μl of the reverse transcription volume was used for each PCR reaction in a total volume of 25 μl. The thermal cycling conditions were the following: 10 min at 95° C., 40 cycles of denaturation at 95° C. for 15 sec and annealing-extension at 60° C. for 1 min. The comparative threshold cycle (CT) method was used for the calculation of amplification fold as specified by the manufacturer. Commercially available primers and probe for GAPDH mRNA were used for normalization (Applied Biosystems, Applera UK, Cheshire, UK). Mammaglobin B mRNA quantities were analyzed in duplicate and mean CT levels were used for further analyses. Results were normalized against GADPH and expressed in relation to a calibrator sample. Results per PCR reaction were expressed as relative gene expression, using the delta-delta CT method [60]. The calibrator was chosen among HOSE and was given a relative expression value of 1.

### Immunohistochemistry on Formalin-Fixed Tissues

To evaluate hMAM-B protein expression level, immunohistochemical staining was performed on 129 samples (i.e. 60 primary tumors, 28 omental metastases, 8 borderline tumors, 26 cystadenomas and 7 normal ovaries, Table 6), stored in the Department of Pathology at the University of Brescia, Italy. Formalin-fixed, paraffin-embedded tissues were cut and stained with H&E and analyzed by a Staff Surgical Pathologist. As controls, surface epithelia obtained from normal ovaries were used. Briefly, formalin-fixed, paraffin-embedded tissues were cut at 2 μm, mounted on charged slide, and dried. For immunohistochemical analysis, slides were deparaffinized and rehydrated in graded solutions of ethanol and distilled water. Endogenous peroxidase was blocked by incubation with peroxidase-blocking solution (cat. #N.S. 2023) (DAKO ChemMate, Calif., USA) for 15 minutes, followed by rinsing in tris-buffered saline (TBS). Non-specific staining was blocked by treatment with normal goat serum (1:50) for 5 minutes. The immunohistochemical method involved sequential application of primary antibody to hMAM diluted 1:50 (Mammaglobin (clone 31A5) Rabbit Monoclonal Antibody, Zeta Corporation, Sierra Madre, Calif., USA) for 45 minutes, a secondary biotinylated anti-rabbit antibody diluted 1:20 (Menarini, Florence, Italy) for 15 minutes and streptavidin-biotin complex diluted 1:20 (Reagent kit, Menarini, Florence, Italy) for 15 minutes. The immunoprecipitate was visualized by treatment with 3′3-diaminobenzidine (Bio-optica, Milan, Italy) for 5 minutes and counterstained by hematoxylin (cat. #N.S. 2020) (DAKO, CA, USA). Immunostaining was considered positive for hMAM-B when at least 10% of neoplastic cells were stained. All samples were scored quantitatively and qualitatively in 20 and 40 high power fields in every section (Nikon, Tokyo, Japan, Eclipse E400). The intensities of hMAM-B expression were blindly scored by 3 independent pathologists from 0 to 3, with grade 0 indicating no staining; grade 1, weak staining; grade 2, moderate staining; and grade 3, strong staining.

### Statistical Analysis

The pairwise difference among histologic types for qRT-PCR and IHC values were tested by means of a multiple nonparametric Behrens-Fisher test procedure [61]. In all the analyses, a P value was considered significant if smaller than 0.05. The correlations between hMAM-B expression measured by qRT-PCR and IHC staining were tested by means of the polyserial correlation coefficient. The polyserial correlation coefficient was computed with the Maximum Likelihood method and the p-value was calculated with a Wald test [62].

### Results

### Mammaplobin B Gene Expression in Ovarian Cancer Tissues

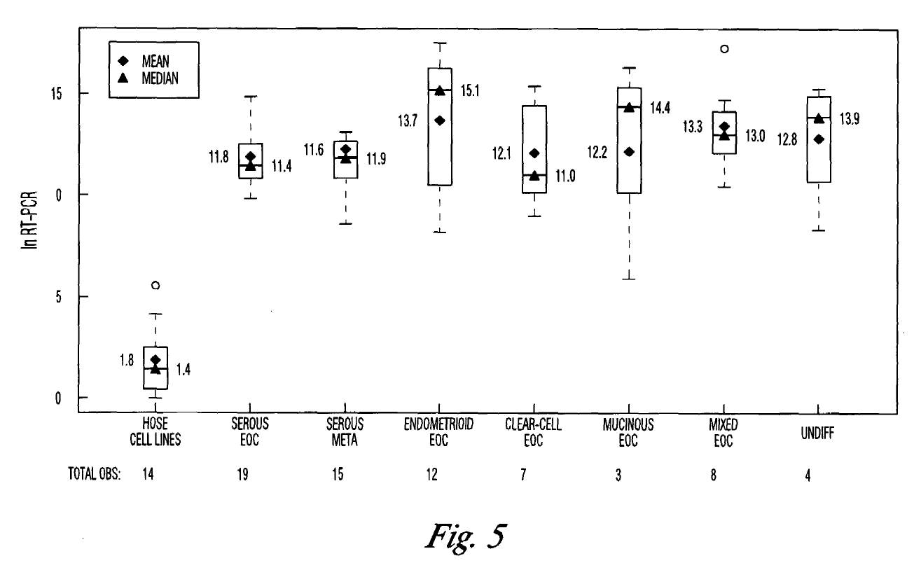

Mammaglobin B gene expression was tested by qRT-PCR in 68 primary and metastatic EOC specimens with various histologies and 14 HOSE primary cell lines. As shown in Table 6 and in FIG. 5, mammaglobin B transcript was detected in all primary EOC biopsies, regardless of histological type, grade and stage of the disease and their primary or metastatic tumor origin. The cutoff point for mammaglobin B mRNA expression was determined as the 95th percentile of the mammaglobin B relative gene expression values of 14 HOSE primary cell lines used as normal controls. Primary and metastatic EOC overexpressed mammaglobin B mRNA at significantly higher levels when compared to HOSE (median copy number by qRT-PCR, 9.33×10⁴ versus 4.00, serous papillary EOC versus HOSE (p<0.01); 3.91×10⁶ versus 4.00, endometrioid EOC versus HOSE (p<0.01); 6.11×10⁴ versus 4.40, clear-cell versus HOSE (p<0.01); 1.73×10⁶ versus 4.00, mucinous EOC versus HOSE (p<0.01); 4.45×10⁵ versus 4.00, mixed EOC versus HOSE (p<0.01); 1.31×10⁶ versus 4.40, undifferentiated EOC versus HOSE (p<0.01); 1.41×10⁵ versus 4.00, serous-papillary metastases versus HOSE (p<0.01) (Table 6). Mammaglobin B expression levels showed variability among primary EOCs belonging to different histological types, however, these differences were not statistically significant.

TABLE 6
qRT-PCR and IHC assay results and polyserial correlation between the two techniques.

| | Mammaglobin B qRT-PCR | | | | | | |
|---|---|---|---|---|---|---|---|
| | | | Inter- | | Mammaglobin | | Polyserial |
| Tissue | | | quartile | # Pos. | B IHC | | correlation |
| Histotype | # tested | Median | range | (%) | # tested | # Pos. (%) | coefficient |
| Normal ovary | 14 | 4 | 9 | 1 (7) | 7 | 0 (0) | — |
| Serous | 0 | — | — | — | 14 | 3 (21) | — |
| cystadenoma | | | | | | | |
| Mucinous | 0 | — | — | — | 12 | 0 (0) | — |
| cystadenoma | | | | | | | |
| Serous-papillary | 19 | 93326 | 24045 | 19 (100) | 20 | 9 (45) | 0.90 |
| EOC | | | | | | | |
| Endometrioid | 12 | 3917866 | 10150582 | 12 (100) | 17 | 13 (76) | 0.76 |
| EOC | | | | | | | |
| Clear-cell EOC | 7 | 61146 | 1838601 | 7 (100) | 8 | 5 (63) | 0.95 |
| Mucinous EOC | 3 | 1739209 | 5931459 | 3 (100) | 3 | 1 (33) | 0.83 |
| Mixed EOC | 8 | 445475 | 869633 | 8 (100) | 8 | 4 (50) | 0.35 |
| Undifferentiated | 4 | 1310720 | 2227200 | 4 (100) | 4 | 1 (25) | 0.00 |
| EOC | | | | | | | |
| Borderline EOC | 0 | — | — | — | 8 | 1 (13) | — |
| Serous-papillary | 15 | 141465 | 254185 | 15 (100) | 28 | 4 (15) | 0.11 |
| Metastases | | | | | | | |

TABLE 7
Mammaglobin B qRT-PCT results according to multiple
nonparametric Behrens-Fisher tests.

| | Adjusted P values |
|---|---|
| Endometrial - HOSE | <0.01 |
| Serous - HOSE | <0.01 |
| Met. Ser. - HOSE | <0.01 |
| Mixed - HOSE | <0.01 |
| Clear-cell - HOSE | <0.01 |
| Undifferentiated - HOSE | <0.01 |
| Mucinous - HOSE | <0.01 |
| Met. Serous - HOSE | 0.21 |
| Serous - Endometrioid | 0.29 |
| Mixed - Met. Serous | 0.58 |
| Serous - Mixed | 0.71 |
| Endometrioid - Clear-cell | 0.71 |
| Mucinous - Endometrioid | 0.92 |
| Mixed - Clear cell | 0.92 |
| Undiff. - Met. Serous | 0.93 |
| Mucinous - Mixed | 0.98 |
| Undiff. - Endom. | 0.98 |
| Undiff. - Clear cell | 1.00 |
| Met. Serous - Clear cell | 1.00 |
| Mucinous - Met. Serous | 1.00 |
| Mixed - Endom. | 1.00 |
| Undiff. - Mucinous | 1.00 |
| Serous - Met. Serous | 1.00 |
| Undiff. - Mixed | 1.00 |
| Serous - Clear cell | 1.00 |
| Serous - Mucinous | 0.95 |
| Mucinous - Clear cell | 1.00 |

### Immunohistochemical Staining for Mammaglobin B

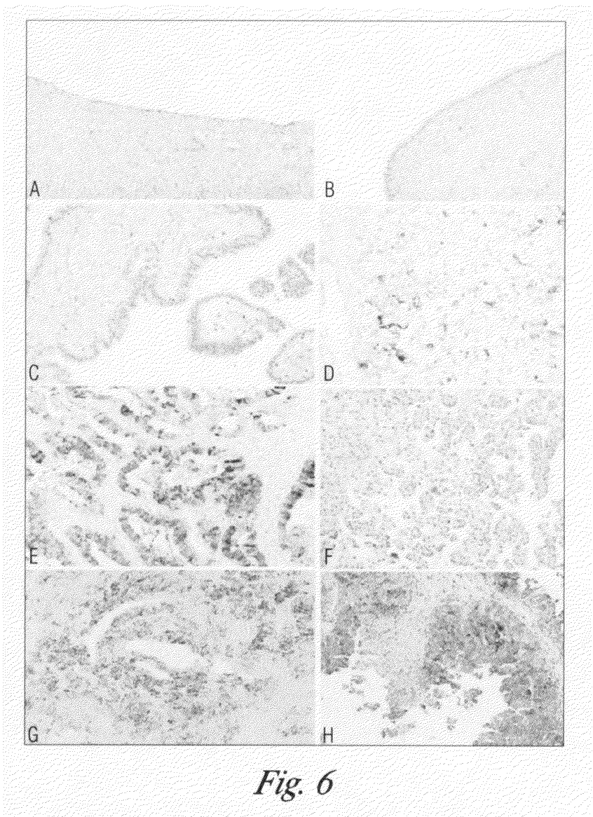

Immunohistochemistry for mammaglobin B protein expression was performed on 60 primary EOC, 28 metastatic serous tumors, 8 borderline ovarian tumors, 26 benign cystadenomas and 7 normal ovaries. As shown in Table 8 mammaglobin B immunoreactivity was detected in 33 (55%) out of 60 primary ovarian cancers, 1 (13%) out of 8 borderline tumors, 4 (14%) out of 28 metastatic cancers and 3 (12%) out of 26 benign cystoadenomas. All normal ovaries tested by IHC were negative for mammaglobin B expression (Table 8, FIG. 6). Reactive stromal cells adjacent to ovarian tumor cells were also found negative in all the pathologic samples analyzed. With the exception of clear cell ovarian cancer, where the staining was limited to the membrane area because of the prominent vacuolization of the cells (FIG. 6, panel D), all other histologic types of ovarian tumors showed diffuse and granular cytoplasmic staining. In agreement with the RT-PCR results, significant differences in mammaglobin B expression were found between normal ovaries and primary ovarian tumors (p<0.01), and between benign cystadenomas and primary ovarian cancers (p<0.01) (Table 9). Of interest, primary OSPC were found to express significantly more mammaglobin B protein when compared to metastatic serous papillary ovarian cancer (p<0.01, Table 9). Mammaglobin B was undetectable in 7 out of 8 borderline tumors (FIG. 6, panel C) while only 3 out of 26 benign cystadenomas showed a weak to moderate mammaglobin B staining (FIG. 6, panels B and F). The correlations between hMAM-B qRT-PCR data and IHC results for each tumoral histotype are illustrated in Table 6.

TABLE 8
Mammaglobin B immunoreactive staining in epithelial ovarian cancer.

| | | Staining Score | | | | |
|---|---|---|---|---|---|---|
| Tissue | Histotype | 3 | 2 | 1 | 0 | Total no. |
| Ovary | Normal | | | | 7 (100%) | 7 |
| Benign | Serous | | 1 (7%) | 2 (14%) | 11 (79%) | 14 |
| cystadenoma | | | | | | |
| | Mucinous | | | | 12 (100) | 12 |
| Primary EOC | Serous-papillary | 2 (10%) | 4 (20%) | 3 (15%) | 11 (55%) | 20 |
| | Endometrioid | 5 (29%) | 7 (41%) | 1 (6%) | 4 (24%) | 17 |
| | Clear-cell | | 4 (50%) | 1 (13%) | 3 (37%) | 8 |
| | Mucinous | | 1 (33%) | | 2 (67%) | 3 |
| | Mixed | 3 (37%) | 1 (13%) | | 4 (50%) | 8 |
| | Undifferentiated | 1 (25%) | | | 3 (75%) | 4 |
| | Borderline | 1 (13%) | | | 7 (87%) | 8 |
| Omental | Serous-papillary | | 1 (4%) | 3 (11%) | 24 (85%) | 28 |
| metastasis | | | | | | |
| Total no. | | 12 | 19 | 10 | 88 | 129 |

TABLE 9
Pairwise comparison in mammaglobin B IHC values
according to multiple nonparametric Behrens-Fisher test.

| | Adjusted P-value |
|---|---|
| Normal ovary - primary tumors | <0.01 |
| Cystadenomas - primary tumors | <0.01 |
| Normal ovary - cystoadenomas | 0.44 |
| Borderline tumors - primary tumors | 0.46 |
| Metastases - normal ovary | 0.86 |
| Borderline tumors - normal ovary | 0.86 |
| Metastasis - cystadenomas | 1.00 |
| Borderline tumors - metastases | 1.00 |
| Borderline tumors - cystadenomas | 1.00 |

### Discussion

In this Example we have quantified mammaglobin B gene expression by qRT-PCR in 53 fresh-frozen primary EOC tissues showing pure (serous, endometrioid, clear-cell, mucinous), mixed and undifferentiated histology. In addition, we have evaluated mammaglobin B transcript expression in several ovarian cancer omental metastases with serous histology. Finally, we have studied mammaglobin B protein expression by IHC on formalin-fixed, paraffin-embedded tissues in a larger cohort of primary and metastatic EOCs.

We found mammaglobin B to be widely expressed in all primary EOCs tested and in serous-papillary metastatic ovarian disease. Indeed, the expression of mammaglobin B in both primary and metastatic ovarian tumors was significantly higher when compared with normal ovary expression (p<0.01), with a median copy number of mammaglobin B gene mRNA 10⁴-10⁶ fold higher in primary EOCs and in metastasis when compared to HOSE cultures. These results, although limited by a relatively low number of clear-cell, mucinous, mixed and undifferentiated ovarian tumors present in our casistic, were confirmed in all histologic types of EOC. Thus, mammaglobin B detection by qRT-PCR is a highly sensitive molecular tool for the identification of primary and secondary ovarian tumor cells with various histological types regardless of the clinical stages and grade of the disease. In this regard, although endometrioid ovarian cancer was found to express the highest levels of mammaglobin B, the variability in expression levels among all different histologic types tested, including serous, clear-cell, mucinous and undifferentiated was not statistically significant (p>0.05). Significant levels of mammaglobin B mRNA were detected in only 1 out of 14 (7%) HOSE control cultures, however its relative expression levels were 10⁴-10⁶ fold lower in normal HOSE controls than in malignant ovarian tissues.

When mammaglobin B protein expression was examined by IHC in EOC we observed immunoreactivity exclusively in tumor cells. Normal ovarian epithelia as well as ovarian stromal cells were found negative for mammaglobin B expression. Of interest, we were able to identify staining in the cytoplasm in all ovarian cancer analyzed that stained positive for mammaglobin B, and, in some cases, we were able to recognize the extrusion of the protein in the apical side of some tumor specimens (data not shown), confirming the secretory nature of mammaglobin B. Only 1 out of 8 (13%) borderline tumors and 3 out of 26 (12%) benign cystadenomas were found to express mammaglobin B. Amongst EOC the majority of IHC positive cases belonged to the endometrioid histologic type followed by the clear-cell and serous-papillary histologic type. Finally, immunoreactive areas in primary tumors with mixed histology mainly referred to their endometrioid component (data not shown). These IHC data were in good correlation with the gene expression results and pairwise comparisons of IHC data were statistically significant between primary tumors and normal ovary, between primary EOCs and benign pathologies and finally between primary and metastatic tumors (p<0.01). Surprisingly however, IHC staining for mammaglobin B performed on serous-papillary metastases gave only 15% immunoreactivity (4 positive cases out of 28). These data contrast with the similar levels of mammaglobin B expression we identified in primary versus metastatic serous papillary ovarian cancer by RT-PCR. These results were further confirmed by investigating mammaglobin B protein expression in 16 matched primary and secondary metastatic serous-papillary EOCs obtained from the same patients. Again only 2 out of 16 metastatic samples (12%) showed mammaglobin B expression when compared to 9 out of 16 (56%) matched primary ovarian tumor tissue.

At this time it is not completely understood why only 42% of EOC samples (29 out of 68) showed positive mammaglobin B immunoreactivity by IHC, although mammaglobin B transcripts were detected in 68 out of 68 (100%) EOC samples tested by qRT-PCR. Other biological mechanisms potentially able to explain the discordance between mRNA and protein abundance in EOC may be related to post-transcriptional mechanisms, including protein translation, post-translational modification and degradation of mammaglobin B in the formalin fixed tumor tissue.

In conclusion, we have demonstrated that mammaglobin B transcript is highly expressed in 100% of the ovarian cancer samples tested regardless of the histologic types, histological grade and clinical stage of the ovarian disease.

### Example 3

### Dendritic Cell-Induced CD8+ Proliferative Responses Against Mammaglobin B Peptides in Healthy Donors

Cryopreserved peripheral blood leukocytes (PBL) from healthy donors are used for generation of dendritic cells (DC). Monocyte-derived DC are cultured in AIM-V (Gibco-BRL) supplemented with GM-CSF and IL-4 [63]. After 5 days' culture, DC maturation is induced by addition of TNFα, IL-1β, and GPE₂ [63]. Mature DC are pulsed for 1-2 hours at 37° C. with 50 μg/ml of whole mammaglobin B, and washed twice before culture with PBL at a responder:stimulatio ratio of 30:1. The culture medium was AIM-V plus 5% human AB serum (Gemini Bioproducts). No IL-2 was added. After 7 days, responder T cells are collected and restimulated with peptide-pulsed DC. For the second and third DC stimulations, the medium is supplemented with 50-100 U/ml IL-2, and the culture period extended to 14 days. After the third cycle, CD8+ T cells are recovered by positive selection with anti-CD8 magnetic beads (Dynal, A. S.). Subsequent restimulations (passages) of CD8+ T cells used peptide-loaded autologous PBL as antigen-presenting cells. CD8+ T cells are quantified after each restimulation, and proliferation of the CD8+ T cells is seen.

### Example 4

### DC-induced CD8+ T Cell Response Against Cells Pulsed with Mammaglobin B Peptides

Dendritic cells are loaded with whole mammaglobin B or a peptide segment of mammaglobin B and used to amplify CD8+ T cells cytotoxic against cells displaying mammaglobin B as described in Example 3. The amplified CD8+ T cell lines are tested for cytoxicity against autologous lymphoblastoid cell lines (LCL) pulsed with the mammaglobin B peptide used to amplify the T cells. Cytotoxicity is tested in a 5 hour ⁵¹Cr-release assay against autologous LCL and autologous LCL pulsed with 50 μg/ml peptide [64, 65]. The amplified CD8+ T cells are found to be cytotoxic to autologous LCL loaded with the mammaglobin B peptide.

### REFERENCES CITED

[1] A. Jemal, T. Murray and E. Ward et al., Cancer statistics, CA Cancer J Clin 55 (2005), pp. 10-30.
[2] R. F. Ozols, Update on the management of ovarian cancer, Cancer J 8 (2002) (Suppl 1), pp. S22-S30.
[3] R. S. Ismail, R. L. Baldwin and J. Fang et al., Differential gene expression between normal and tumor-derived ovarian epithelial cells, Cancer Res 60 (2000), pp. 6744-6749.
[4] J. B. Welsh, P. P. Zarrinkar and L. M. Sapinoso et al., Analysis of gene expression profiles in normal and neoplastic ovarian tissue samples identifies candidate molecular markers of epithelial ovarian cancer, Proc Natl Acad Sci USA 98 (2001), pp. 1176-1181.
[5] D. R. Schwartz, S. L. Kardia and K. A. Shedden et al., Gene expression in ovarian cancer reflects both morphology and biological behavior, distinguishing clear cell from other poor-prognosis ovarian carcinomas, Cancer Res 62 (2002), pp. 4722-4729.
[6] C. D. Hough, C. A. Sherman-Baust and E. S. Pizer et al., Large-scale serial analysis of gene expression reveals genes differentially expressed in ovarian cancer, Cancer Res 60 (2000), pp. 6281-6287.
[7] K. Ono, T. Tanaka and T. Tsunoda et al., Identification by cDNA microarray of genes involved in ovarian carcinogenesis, Cancer Res 60 (2000), pp. 5007-5011.
[8] V. Shridhar, J. Lee and A. Pandita et al., Genetic analysis of early- versus late-stage ovarian tumors, Cancer Res 61 (2001), pp. 5895-5904.
[9] C. D. Hough, K. R. Cho, A. B. Zonderman, D. R. Schwartz and P. J. Morin, Coordinately up-regulated genes in ovarian cancer, Cancer Res 6 (2001), pp. 3869-3876.
[10] V. Shridhar, A. Sen and J. Chien et al., Identification of underexpressed genes in early- and late-stage primary ovarian tumors by suppression subtraction hybridization, Cancer Res 62 (2002), pp. 262-270.
[11] A. A. Jazaeri, K. Lu and R. Schmandt et al., Molecular determinants of tumor differentiation in papillary serous ovarian carcinoma, Mol Carcinog 36 (2003), pp. 53-59.
[12] M. E. Schaner, D. T. Ross and G. Ciaravino et al., Gene expression patterns in ovarian carcinomas, Mol Biol Cell 14 (2003), pp. 4376-4386.
[13] T. R. Adib, S. Henderson and C. Perrett et al., Predicting biomarkers for ovarian cancer using gene-expression microarrays, Br J Cancer 9 (2004) (90), pp. 686-692.
[14] A. D. Santin, F. Zhan and S. Bellone et al., Gene expression profiles in primary ovarian serous papillary tumors and normal ovarian epithelium: identification of candidate molecular markers for ovarian cancer diagnosis and therapy, Int J Cancer 20 (2004) (112), pp. 14-25.
[15] H. Donninger, T. Bonome and M. Radonovich et al., Whole genome expression profiling of advance stage papillary serous ovarian cancer reveals activated pathways, Oncogene 21 (2004) (23), pp. 8065-8077.
[16] D. Spentzos, D. A. Levine and M. F. Ramoni et al., Gene expression signature with independent prognostic significance in epithelial ovarian cancer, J Clin Oncol 22 (2004), pp. 4648-4658.
[17] K. M. Feeley and M. Wells, Precursor lesions of ovarian epithelial malignancy, Histopathology 38 (2001) (2), pp. 87-95.
[18] A. Berchuck, E. S. Iversen and J. M. Lancaster et al., Prediction of optimal versus suboptimal cytoreduction of advanced-stage serous ovarian cancer with the use of microarrays, Am J Obstet Gynecol 190 (2004), pp. 910-925.
[19] K. Hibbs, K. M. Skubitz and S. E. Pambuccian et al., Differential gene expression in ovarian carcinoma: identification of potential biomarkers, Am J Pathol 165 ((August)2004) (2), pp. 397-414.
Schummer M, Ng W V, Bumgarner R E, et al. Comparative hybridization of an array of 21,500 ovarian cDNAs for the discovery of genes overexpressed in ovarian carcinomas. Gene 1999; 1;238(2):375-85.
[21] K. K. Zorn, A. A. Jazaeri and C. S. Awtrey et al., Choice of normal ovarian control influences determination of differentially expressed genes in ovarian cancer expression profiling studies, Clin Cancer Res 9 (2003), pp. 4811-4818.
[22] A. D. Santin, F. Zhan and E. Bignotti et al., Gene expression profiles of primary HPV 16- and HPV18-infected early stage cervical cancers and normal cervical epithelium: identification of novel candidate molecular markers for cervical cancer diagnosis and therapy, Virology 331 (2005), pp. 269-291.
[23] Affymetrix Statistical Algorithms Description Document, Affymetrix Inc., Santa Clara, Calif., whitepaper, 2002.
[24] V. G. Tusher, R. Tibshirani and G. Chu, Significance analysis of microarrays applied to the ionizing radiation response, PNAS 98 (2001), pp. 5116-5121.
[25] B. Efron, R. Tibshirani, J. D. Storey and V. Tusher, Empirical bayes analysis of a microarray experiment, JASA (2001), pp. 1151-1160.
[26] R Development Core Team. R: A language and environment for statistical computing. R Foundation for Statistical Computing, Vienna, Austria (2005). ISBN 3-900051-07-0, URL http://www.R-project.org.
[27] R. C. Gentleman, V. J. Carey and D. M. Bates et al., Bioconductor: open software development for computational biology and bioinformatics, Genome Biol 5 (2004) (10), p. R8.
[28] F. Muller-Schottle, I. Classen-Linke, K. Beier-Hellwig, K. Sterzik and H. M. Beier, Uteroglobin expression and release in the human endometrium, Ann NY Acad Sci 923 (2000), pp. 332-335.
[29] R. M. Becker, C. Darrow, D. B. Zimonjic, N. C. Popescu, M. A. Watson and T. P. Fleming, Identification of mammaglobin B, a novel member of the uteroglobin gene family, Genomics 15 (1998) (54), pp. 70-78.
[30] T. Aihara, Y. Fujiwara and Y. Miyake et al., Mammaglobin B gene as a novel marker for lymph node micrometastasis in patients with abdominal cancers, Cancer Lett 13 ((March) 2000) (150), pp. 79-84.
[31] R. J. Ouellette, D. Richard and E. Maicas, RT-PCR for mammaglobin genes, MGB1 and MGB2, identifies breast cancer micrometastases in sentinel lymph nodes, Am J Clin Pathol 121 (2004), pp. 637-643.
[32] M. Ooka, I. Sakita and Y. Fujiwara et al., Selection of mRNA markers for detection of lymph node micrometastases in breast cancer patients, Oncol Rep 7 (2000), pp. 561-566.
[33] E. P. Diamandis and G. M. Yousef, Human tissue kallikreins: a family of new cancer biomarkers, Clin Chem 48 (2002), pp. 1198-1205.
[34] C. A. Borgoño and E. P. Diamandis, The emerging roles of human tissue kallikreins in cancer, Nat Rev Cancer 4 (2004), pp. 876-890.
[35] G. M. Yousef, M. E. Polymeris and G. M. Yacoub et al., Parallel overexpression of seven kallikrein genes in ovarian cancer, Cancer Res 63 (2003) (1), pp. 2223-2227.
[36] Diamandis E P, Scorilas A, Fracchioli S, et al. Human kallikrein 6 (hK6): a new potential serum biomarker for diagnosis and prognosis of ovarian carcinoma. J Clin Oncol 2003;15;21(6):1035-43.
[37] M. J. Cannon, T. J. O'Brien, L. J. Underwood, M. D. Crew, K. L. Bondurant and A. D. Santin, Novel target antigens for dendritic cell-based immunotherapy against ovarian cancer, Exp Rev Anticancer Ther 2 (2002), pp. 97-105.
[38] K. L. Bondurant, M. D. Crew, A. D. Santin, T. J. O'Brien and M. J. Cannon, Definition of an immunogenic region within the ovarian tumor antigen stratum corneum chymotryptic enzyme, Clin Cancer Res 11 (2005), pp. 3446-3454.
[39] B. A. McClane, An overview of clostridium perfringens enterotoxin, Toxicon 34 (1996), pp. 1335-1343.
[40] L. B. Rangel, R. Agarwal and T. D'Souza et al., Tight junction proteins claudin-3 and claudin-4 are frequently overexpressed in ovarian cancer but not in ovarian cystadenomas, Clin Cancer Res 9 (2003), pp. 2567-2575.
[41] H. Long, C. D. Crean, W. H. Lee, O. W. Cummings and T. G. Gabig, Expression of Clostridium perfringens enterotoxin receptors claudin-3 and claudin-4 in prostate cancer epithelium, Cancer Res 61 (2001), pp. 7878-7881.
[42] P. Michl, M. Buchholz and M. Rolke et al., Claudin-4: a new target for pancreatic cancer treatment using clostridium perfringens enterotoxin, Gastroenterology 121 (2001), pp. 678-684.
[43] A. D. Santin, S. Cane and S. Bellone et al., Treatment of chemotherapy-resistant human ovarian cancer xenografts in C.B-17/SCID mice by intraperitoneal administration of clostridium perfringens enterotoxin, Cancer Res 65 (2005), pp. 4334-4342.
[44] B. M. Carreno and M. Collins, The B7 family of ligands and its receptors: new pathways for costimulation and inhibition of immune responses, Annu Rev Immunol 20 (2002), pp. 29-53.
[45] S. J. Khoury and M. H. Sayegh, The roles of the new negative T cell costimulatory pathways in regulating autoimmunity, Immunity 20 (2004), pp. 529-538.
[46] X. Zang, P. Loke, J. Kim, K. Murphy, R. Waitz and J. P. Allison, B7x: a widely expressed B7 family member that inhibits T cell activation, Proc Natl Acad Sci USA 100 (2003), pp. 10388-10392.
[47] D. V. Prasad, S. Richards, X. M. Mai and C. Dong, B7S1, a novel B7 family member that negatively regulates T cell activation, Immunity 18 (2003), pp. 863-873.
[48] G. L. Sica, I. H. Choi and G. Zhu et al., B7-H4, a molecule of the B7 family, negatively regulates T cell immunity, Immunity 18 (2003), pp. 849-861.
[49] S. Salceda, T. Tang and M. Kmet et al., The immunomodulatory protein B7-H4 is overexpressed in breast and ovarian cancers and promotes epithelial cell transformation, Exp Cell Res 306 (2005), pp. 128-141.
[50] Y. Liu and T. S. Ganesan, Tumour suppressor genes in sporadic epithelial ovarian cancer, Reproduction 123 (2002), pp. 341-353.
[51] Mercatali L, Valenti V, Calistri D, Calpona S, Rosti G, Folli S, Gaudio M, Frassineti G L, Amadori D, Flamini E. RT-PCR determination of maspin and mammaglobin B in peripheral blood of healthy donors and breast cancer patients. Ann Oncol. March 2006; 17(3):424-8.
[52] Nissan A, Jager D, Roystacher M, Prus D, Peretz T, Eisenberg I, Freund H R, Scanlan M, Ritter G, Old L J, Mitrani-Rosenbaum S. Multimarker RT-PCR assay for the detection of minimal residual disease in sentinel lymph nodes of breast cancer patients. Br J Cancer 2006; 94:681-5
[53] Ooka M, Sakita I, Fujiwara Y, Tamaki Y, Yamamoto H, Aihara T, Miyazaki M, Kadota M, Masuda N, Sugita Y, Iwao K, Monden M. Selection of mRNA markers for detection of lymph node micrometastases in breast cancer patients. Oncol Rep 2000; 7:561-6.
[54] O'Brien N, Maguire T M, O'Donovan N, Lynch N, Hill A D, McDermott E, O'Higgins N, Duffy M J. Mammaglobin a: a promising marker for breast cancer. Clin Chem 2002; 48:1362-4.
[55] Santin A D, Zhan F, Bellone S, Palmieri M, Cane S, Bignotti E, Anfossi S, Gokden M, Dunn D, Roman J J, O'Brien T J, Tian E, Cannon M J, Shaughnessy J Jr, Pecorelli S. Gene expression profiles in primary ovarian serous papillary tumors and normal ovarian epithelium: identification of candidate molecular markers for ovarian cancer diagnosis and therapy. Int J Cancer. 2004; 112:14-25.
[56] Livak K J and Schmittgen T D. Analysis of relative gene expression data using real-time quantitative PCR and the 2(-Delta Delta C(T)) Method. Methods 2001; 25:402-08.
[57] Munzel U and Hothom LA. A unified approach to simultaneous rank test procedures in the unbalanced one-way layout. Biometrical J 2001; 5: 553-69.
[58] Drasgow F. Polychoric and polyserial correlations. In: S. Kotz and N. Johnson, editors. The Encyclopedia of Statistics, Volume 7. Wiley; 1986. p. 68-74.
[59] O'Brien N, Maguire T M, O'Donovan N, Lynch N, Hill A D, McDermott E, O'Higgins N, Duffy M J. Mammaglobin a: a promising marker for breast cancer. Clin Chem 2002; 48:1362-4.
[60] Livak K J and Schmittgen T D. Analysis of relative gene expression data using real-time quantitative PCR and the 2(-Delta Delta C(T)) Method. Methods 2001; 25:402-08.
[61] Munzel U and Hothorn L A. A unified approach to simultaneous rank test procedures in the unbalanced one-way layout. Biometrical J 2001; 5: 553-69.
[62] Drasgow F. Polychoric and polyserial correlations. In: S. Kotz and N. Johnson, editors. The Encyclopedia of Statistics, Volume 7. Wiley; 1986. p. 68-74.
[63] Santin A D. Hermonat P L. Ravaggi A. Bellone S. Roman J J. Jayaprabhu S. Pecorelli S. Parham G P. Cannon M J. (2001) Expression of CD56 by human papillomavirus E7-specific CD8+ cytotoxic T lymphocytes correlates with increased intracellular perforin expression and enhanced cytotoxicity against HLA-A2-matched cervical tumor cells. Clin Cancer Res. 7(3 Suppl):804s-810s.
[64] Levitsky V, Zhang Q J, Levitskaya J, Masucci M. G. (1996) The life span of major histocompatibility complex-peptide complexes influences the efficiency of presentation and immunogenicity of two class I-restricted cytotoxic T lymphocyte epitopes in the Epstein-Barr virus nuclear antigen 4. J Exp Med. 183(3):915-26.
[65] Torsteinsdottir S, Masucci M G, Ehlin-Henriksson B, Brautbar C, Ben Bassat H, Klein G, Klein E. (1986) Differentiation-dependent sensitivity of human B-cell-derived lines to major histocompatibility complex-restricted T-cell cytotoxicity. Proc Natl Acad Sci USA. 83(15):5620-4.
[66] Current Protocols in Immunology, 2007, Wiley InterScience.
[67]. Wands et al. 1981. Gastroenterology 80:225-232.
[68] Muller-Schottle F, Classen-Linke I, Beier-Hellwig K, Sterzik K, Beier H M. Uteroglobin expression and release in the human endometrium. Ann NY Acad Sci 2000;923:332-35.
[69] Aihara T, Fujiwara Y, Ooka M, Sakita I, Tamaki Y, Monden M. Mammaglobin B as a novel marker for detection of breast cancer micrometastases in axillary lymph nodes by reverse transcription-polymerase chain reaction. Breast Cancer Res Treat. 1999; 58:137-40.
[70] Ouellette R J, Richard D, Maicas E. RT-PCR for mammaglobin genes, MGB1 and MGB2, identifies breast cancer micrometastases in sentinel lymph nodes. Am J Clin Pathol. May 2004; 121:637-43.
[71] Banchereau J, Steinman R M. Dendritic cells and the control of immunity. Nature 1998; 392:245-252.
[72] Mellman, I. et al., 2001, Dendritic cells: specialized antigen-presenting machines. Cell 106:255-258.
[73] Kohler et al., Nature 256:495 (1975)
[74] Kohler et al., Eur. J. Immunol. 6:511 (1976);
[75] Kohler et al., Eur. J. Immunol. 6:292 (1976).
[76] Hammerling et al., in: Monoclonal Antibodies and T-Cell Hybridomas, Elsevier, N.Y., pp. 563-681 (1981).
[77] Morrison, Science 229:1202 (1985)
[78] Oi et al., BioTechniques 4:214 (1986).
[79] Cabilly et al., U.S. Pat. No. 4,816,567.
[80] Taniguchi et al., EP 171496
[81] Morrison et al., EP 173494
[82] Neuberger et al., WO 8601533
[83] Robinson et al., WO 8702671.
[84] Boulianne et al., Nature 312:643 (1984).
[85] Neuberger et al., Nature 314:268 (1985).

All patents, patent applications, and other references cited are incorporated by reference.

What is claimed is:

1. A method of screening for cancer comprising:
obtaining a fluid sample from a mammal;
contacting the fluid sample with a mammaglobin B binding protein to bind mammaglobin B in the sample; and
quantifying binding of the binding protein to mammaglobin B to detect the presence of mammaglobin B above a threshold level, wherein the presence of mammaglobin B above the threshold level indicates presence of cancer in the mammal.

2. The method of claim 1 wherein the cancer is a gynecological malignancy.

3. The method of claim 2 wherein the gynecological malignancy is ovarian cancer.

4. The method of claim 1 wherein the fluid sample is blood or ascites fluid.

5. The method of claim 1 wherein the cancer has not metastasized.

6. A kit to detect mammaglobin B in a biological fluid sample comprising:
a mammaglobin B binding protein; and
a means for detecting binding of the mammaglobin B binding protein to mammaglobin B in a biological fluid sample.

7. A method of treating cancer comprising:
inoculating a mammal suffering from or at risk of cancer with a mammaglobin B peptide, wherein the inoculation elicits an immune response in the mammal against cells expressing mammaglobin B.

8. The method of claim 7 wherein the peptide is on dendritic cells.

9. The method of claim 7 wherein the peptide is associated with an adjuvant.

10. The method of claim 7 wherein the inoculating slows the growth of the cancer.

11. The method of claim 7 wherein the mammal is in remission from cancer and the inoculating decreases the risk of recurrence of the cancer.

12. The method of claim 7 wherein the cancer is a gynecological malignancy.

13. The method of claim 12 wherein the cancer is ovarian cancer.

14. An immunogenic composition comprising a mammaglobin B peptide and an adjuvant.

15. An immunogenic composition comprising dendritic cells loaded with a mammaglobin B peptide.
